SEQUENCE LISTING (47 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 1154 aa, Artificial Sequence
  recombinant spider silk protein ADF3KaiLargeNRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 5 - protein, 24 aa, Artificial Sequence
  His tag and start codon
MHHHHHHHHHHSSGSSLEVLFQGP

SEQ ID NO: 6 - protein, 597 aa, Artificial Sequence
  Met-PRT380
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 7 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 8 - protein, 565 aa, Artificial Sequence
  Met-PRT525
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 9 - protein, 2364 aa, Artificial Sequence
  Met-PRT799
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 10 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 11 - protein, 12 aa, Artificial Sequence
  HisTag
MHHHHHHSSGSS

SEQ ID NO: 12 - protein, 608 aa, Artificial Sequence
  PRT380
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 13 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 14 - protein, 576 aa, Artificial Sequence
  PRT525
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 15 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 16 - protein, 608 aa, Artificial Sequence
  PRT313
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 17 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 18 - protein, 601 aa, Artificial Sequence
  PRT399
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 19 - protein, 612 aa, Artificial Sequence
  Met-PRT720
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 20 - protein, 592 aa, Artificial Sequence
  Met-PRT665
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 21 - protein, 619 aa, Artificial Sequence
  Met-PRT666
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 22 - protein, 623 aa, Artificial Sequence
  PRT720
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 23 - protein, 603 aa, Artificial Sequence
  PRT665
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 24 - protein, 630 aa, Artificial Sequence
  PRT666
MHHHHHHSSGSSGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 25 - protein, 593 aa, Artificial Sequence
  M_PRT888
MGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 26 - protein, 590 aa, Artificial Sequence
  M_PRT965
MGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 27 - protein, 593 aa, Artificial Sequence
  M_PRT889
MGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 28 - protein, 590 aa, Artificial Sequence
  M_PRT916
MGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 29 - protein, 590 aa, Artificial Sequence
  M_ PRT918
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 30 - protein, 565 aa, Artificial Sequence
  M_PRT699
MGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 31 - protein, 565 aa, Artificial Sequence
  M_PRT698
MGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 32 - protein, 2360 aa, Artificial Sequence
  Met-PRT1009
GPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASEFGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASELGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 33 - protein, 601 aa, Artificial Sequence
  PRT888
MHHHHHHSSGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 34 - protein, 601 aa, Artificial Sequence
  PRT965
MHHHHHHSSGSSGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 35 - protein, 601 aa, Artificial Sequence
  PRT889
MHHHHHHSSGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 36 - protein, 601 aa, Artificial Sequence
  PRT916
MHHHHHHSSGSSGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 37 - protein, 601 aa, Artificial Sequence
  PRT918
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 38 - protein, 576 aa, Artificial Sequence
  PRT699
MHHHHHHSSGSSGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 39 - protein, 576 aa, Artificial Sequence
  PRT698
MHHHHHHSSGSSGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 40 - protein, 2372 aa, Artificial Sequence
  PRT1009
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASEFGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASELGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 41 - protein, 559 aa, Artificial Sequence
  PRT215
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 42 - protein, 252 aa, Artificial Sequence
  Collagen-type4-Kai
MHHHHHHSSGSSKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEK

SEQ ID NO: 43 - protein, 310 aa, Artificial Sequence
  Resilin-Kai
MHHHHHHPEPPVNSYLPPSDSYGAPGQSGPGGRPSDSYGAPGGGNGGRPSDSYGAPGQGQGQGQGQGGYAGKPSDSYGAPGGGDGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASG

SEQ ID NO: 44 - protein, 282 aa, Artificial Sequence
  elastin short
MHHHHHHSSGSSLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVG

SEQ ID NO: 45 - protein, 487 aa, Artificial Sequence
  PRT798
MHHHHHHSSGSSLEVLFQGPSFRLSGVSRRLCSQAGTGRLTGGRTGFRAGNVCSGLGAGSSFSGPLGSVSSKGSFSHGGGGLGSGVCTGFLENEHGLLPGNEKVTLQNLNDRLASYLDHVCTLEEANADLEQKIKGWYEKYGPGSGRQLAHDYSKYFSVTEDLKRQIISVTTCNASIVLQNENARLTADDFRLKCENELALHQSVEADINGLHRVMDELTLCTSDLEMQCEALSEELTYLKKNHQEEMKVMQGAARGNVNVEINAAPGVDLTVLLNNMRAEYEDLAEQNHEDAEAWFSEKSTSLHQQISDDAGAAMAARNELMELKRNLQTLEIELQSLLAMKHSYECSLAETESNYCHQLQQIQEQIGAMEDQLQQIRMETEGQKLEHERLLDVKIFLEKEIEMYCKLIDGEGRKSKSTCYKSEGRGPKNSENQVKDSKEEAVVKTVVGELDQLGSVLSLRVHSVEEKSSKISNITMEQRLPSKVP

SEQ ID NO: 46 - protein, 1179 aa, Artificial Sequence
  Met-PRT966
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 47 - protein, 1190 aa, Artificial Sequence
  PRT966
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

(19) United States
(12) Patent Application Publication
(10) Pub. No.: US 2021/0095399 A1
(43) Pub. Date: Apr. 1, 2021

(54) Composite Fibers and Method for Manufacturing Same

(71) Applicant: Spiber Inc., Tsuruoka-shi, Yamagata (JP)

(72) Inventors: Yunosuke Abe, Tsuruoka-shi, Yamagata (JP); Shota Togashi, Tsuruoka-shi, Yamagata (JP)

(73) Assignee: Spiber Inc., Tsuruoka-shi, Yamagata (JP)

(21) Appl. No.: 17/043,834

(22) PCT Filed: Apr. 3, 2019
(86) PCT No.: PCT/JP2019/014867

§ 371 Date: Oct. 29, 2020

(30) Foreign Application Priority Data
Apr. 3, 2018 (JP) 2018-071955

(51) Int. Cl. D01F 8/02; D01F 4/02; D01D 5/32

(52) U.S. Cl. CPC D01F 8/02; D01F 4/02; D10B 2211/22; D10B 2211/04; D01D 5/32

(57) ABSTRACT

Provided is a side-by-side type composite fiber having a latent crimping ability, including a first component containing a modified fibroin and a second component containing a structural protein, in which the first component and the second component are joined to each other.

### TECHNICAL FIELD

The present invention relates to a composite fiber and a method for manufacturing the composite fiber.

### BACKGROUND ART

Crimping ability is a very important property for performing spinning. In the case of thermoplastic synthetic fibers such as nylon and a polyester fiber, it is possible to manufacture a fiber having permanently crimping ability by utilizing heat. In addition, raw silk obtained from silkworm is covered with sericin and is converted into a silk fiber (fibroin) which is soft and glossy by a scouring process.

On the other hand, a silk fiber from which sericin has been completely removed has no elasticity and is not suitable for a twisted yarn. Therefore, in order to manufacture a twisted yarn from a silk fiber, a method in which scouring is performed, without completely removing sericin, such that 80% to 90% of sericin remains to maintain an appropriate elasticity is used. In addition, various contrivances have been made, such as carrying out the scouring process at the yarn stage, carrying out the scouring process at the textile stage, not carrying out the scouring process, and weaving a sericin-fixed yarn and a sericin-unfixed yarn (For example, Patent Literature 1 and Non-Patent Literature 1). In recent years, a method in which sericin is insolubilized with a modifier without removing sericin and twisting process is performed has been reported (for example, Patent Literature 2).

### CITATION LIST

### Patent Literature

[Patent Literature 1] Japanese Unexamined Patent Publication No. H5-93317
[Patent Literature 2] Japanese Unexamined Patent Publication No. 2016-23389

### Non Patent Literature

[Non-Patent Literature 1] Ling Peng et al. Macromol. Mater. Eng. 2016, 301, 48-55.

### SUMMARY OF INVENTION

### Technical Problem

However, it was necessary to adjust the content rate of sericin to the desired range.

In consideration of the above circumstances, an object of the present invention is to provide a new fiber having crimping ability and a method for manufacturing the fiber having crimping ability.

### Solution to Problem

The present invention relates to, for example, each of the following inventions.

[1] A side-by-side type composite fiber having a latent crimping ability, including a first component containing a modified fibroin and a second component containing a structural protein, in which the first component and the second component are joined to each other.

[2] The composite fiber according to [1], in which a composition ratio of the first component to the second component is 9:1 to 1:9 based on a mass of the composite fiber.

[3] The composite fiber according to [1] or [2], in which the structural protein is at least one selected from the group consisting of a silk fibroin, a spider silk fibroin, collagen, resilin, elastin, and keratin.

[4] The composite fiber according to any of [1] to [3], in which the structural protein is at least one selected from the group consisting of a silk fibroin, a spider silk fibroin, and keratin.

[4-1] The composite fiber according to any of [1] to [4], in which the modified fibroin is a modified fibroin (a third modified fibroin) including a domain sequence represented by Formula 1: [(A)ₙ motif−REP]ₘ, and

the domain sequence has an amino acid sequence in which a content of the (A)ₙ motif is reduced, the amino acid sequence being equivalent to an amino acid sequence in which at least one or a plurality of the (A)ₙ motifs are deleted, as compared with a naturally occurring fibroin.

[In Formula 1, the (A)ₙ motif represents an amino acid sequence composed of 2 to 27 amino acid residues and the number of alanine residues with respect to a total number of amino acid residues in the (A)ₙ motif is 83% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[4-2] The composite fiber according to any of [1] to [4], in which the modified fibroin is a modified fibroin (a fourth modified fibroin) including a domain sequence represented by Formula 1: [(A)ₙ motif−REP]ₘ, and

the domain sequence has an amino acid sequence in which a content of a glycine residue is reduced, the amino acid sequence being equivalent to an amino acid sequence in which at least one or a plurality of the glycine residues in the REP are substituted with other amino acid residues, as compared with a naturally occurring fibroin.

[In Formula 1, the (A)ₙ motif represents an amino acid sequence composed of 2 to 27 amino acid residues and the number of alanine residues with respect to a total number of amino acid residues in the (A)ₙ motif is 83% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[4-3] The composite fiber according to any of [1] to [4], in which the modified fibroin is a modified fibroin (a fifth modified fibroin) including a domain sequence represented by Formula 1: [(A)ₙ motif−REP]ₘ, and

the domain sequence has an amino acid sequence locally including a region having a high hydropathy index, the amino acid sequence being equivalent to an amino acid sequence in which one or a plurality of amino acid residues in the REP are substituted with amino acid residues having a high hydropathy index and/or one or a plurality of amino acid residues having a high hydropathy index are inserted into the REP, as compared with a naturally occurring fibroin.

[In Formula 1, the (A)ₙ motif represents an amino acid sequence composed of 2 to 27 amino acid residues and the number of alanine residues with respect to a total number of amino acid residues in the (A)ₙ motif is 83% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[4-4] The composite fiber according to any of [1] to [4], in which the modified fibroin a modified fibroin (a sixth modified fibroin) including a domain sequence represented by Formula 1: [(A)ₙ motif−REP]ₘ or Formula 2: [(A)ₙ motif−REP]ₘ−(A)ₙ motif, and

the domain sequence has an amino acid sequence in which a content of a glutamine residue is reduced, the amino acid sequence being equivalent to an amino acid sequence in which one or a plurality of the glutamine residues in the REP are deleted or substituted with other amino acid residues, as compared with a naturally occurring fibroin.

[In Formula 1 and Formula 2, the (A)ₙ motif represents an amino acid sequence composed of 2 to 27 amino acid residues and the number of alanine residues with respect to a total number of amino acid residues in the (A)ₙ motif is 80% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[4-5] The composite fiber according to any one of [1] to [4], in which the modified fibroin has a limiting oxygen index (LOI) value of 26.0 or more.

[4-6] The composite fiber according to any of [1] to [4], in which the modified fibroin has a highest hygroscopic heat generation degree of more than 0.025° C./g, the highest hygroscopic heat generation degree being determined according to Expression A.

highest hygroscopic heat generation degree={(highest temperature of a sample when the sample has been transferred to a high humidity environment after being placed in a low humidity environment until a temperature of the sample reaches equilibrium)−(temperature of the sample when the sample is being transferred to the high humidity environment after being placed in the low humidity environment until the temperature of the sample reaches equilibrium)}(° C.)/sample weight (g) Expression A:

[In Expression A, the low humidity environment means an environment of a temperature of 20° C. and a relative humidity of 40%, and the high humidity environment means an environment of a temperature of 20° C. and a relative humidity of 90%.]

[5] A method for manufacturing a composite fiber, comprising:

preparing a first doping liquid containing a modified fibroin and a solvent;

preparing a second doping liquid containing a structural protein and a solvent;

discharging the first doping liquid and the second doping liquid from a spinneret and joining the first doping liquid and the second doping liquid to form an undrawn composite fiber in a coagulation liquid; and

bringing the undrawn composite fiber into contact with an aqueous medium.

[6] The method according to [5], further including drawing the undrawn composite fiber.

[7] The method according to [5] or [6], in which a concentration of the modified fibroin in the first doping liquid is 5% to 40% by mass based on a total mass of the first doping liquid, and

a concentration of the structural protein in the second doping liquid is 5% to 40% by mass based on a total mass of the second doping liquid.

[8] The method according to any of [5] to [7], in which the solvent contains at least one selected from the group consisting of hexafluoroisopropanol, hexafluoroacetone, dimethyl sulfoxide, N,N-dimethylformamide, N,N-dimethylacetamide, 1,3-dimethyl-2-imidazolidone, N-methyl-2-pyrrolidone, acetonitrile, N-methylmorpholine-N-oxide, formic acid, and an aqueous solution containing at least one selected from the group consisting of urea, guanidine, sodium dodecyl sulfate, lithium bromide, calcium chloride, and lithium thiocyanate.

[9] The method according to any of [5] to [8], in which the coagulation liquid is at least one selected from the group consisting of a lower alcohol having 1 to 5 carbon atoms and acetone.

[10] The method according to any of [5] to [9], in which the structural protein is at least one selected from the group consisting of a silk fibroin, a spider silk fibroin, collagen, resilin, elastin, and keratin. [11] The method according to any of [5] to [10], in which the structural protein is at least one selected from the group consisting of a silk fibroin, a spider silk fibroin, and keratin.

[11-1] The method according to any of [5] to [11], in which the modified fibroin is a modified fibroin including a domain sequence represented by Formula 1: [(A)ₙ motif−REP]ₘ, and

the domain sequence has an amino acid sequence in which a content of the (A)ₙ motif is reduced, the amino acid sequence being equivalent to an amino acid sequence in which at least one or a plurality of the (A)ₙ motifs are deleted, as compared with a naturally occurring fibroin.

[In Formula 1, the (A)ₙ motif represents an amino acid sequence composed of 2 to 27 amino acid residues and the number of alanine residues with respect to a total number of amino acid residues in the (A)ₙ motif is 83% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[11-2] The method according to any of [5] to [11], in which the modified fibroin includes a domain sequence represented by Formula 1: [(A)ₙ motif−REP]ₘ, and

the domain sequence has an amino acid sequence in which a content of a glycine residue is reduced, the amino acid sequence being equivalent to an amino acid sequence in which at least one or a plurality of the glycine residues in the REP are substituted with other amino acid residues, as compared with a naturally occurring fibroin.

[In Formula 1, the (A)ₙ motif represents an amino acid sequence composed of 2 to 27 amino acid residues and the number of alanine residues with respect to a total number of amino acid residues in the (A)ₙ motif is 83% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[11-3] The method according to any of [5] to [11], in which the modified fibroin includes a domain sequence represented by Formula 1: [(A)ₙ motif−REP]ₘ, and

the domain sequence has an amino acid sequence locally including a region having a high hydropathy index, the amino acid sequence being equivalent to an amino acid sequence in which one or a plurality of amino acid residues in the REP are substituted with amino acid residues having a high hydropathy index and/or one or a plurality of amino acid residues having a high hydropathy index are inserted into the REP, as compared with a naturally occurring fibroin.

[In Formula 1, the (A)ₙ motif represents an amino acid sequence composed of 2 to 27 amino acid residues and the number of alanine residues with respect to a total number of amino acid residues in the (A)ₙ motif is 83% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[11-4] The method according to any of [5] to [11], in which the modified fibroin includes a domain sequence represented by Formula 1: [(A)ₙ motif−REP]ₘ or Formula 2: [(A)ₙ motif−REP]ₘ−(A)ₙ motif, and

the domain sequence has an amino acid sequence in which a content of a glutamine residue is reduced, the amino acid sequence being equivalent to an amino acid sequence in which one or a plurality of the glutamine residues in the REP are deleted or substituted with other amino acid residues, as compared with a naturally occurring fibroin.

[In Formula 1 and Formula 2, the (A)ₙ motif represents an amino acid sequence composed of 2 to 27 amino acid residues and the number of alanine residues with respect to a total number of amino acid residues in the (A)ₙ motif is 80% or more. The REP represents an amino acid sequence composed of 10 to 200 amino acid residues. m represents an integer of 2 to 300. A plurality of the (A)ₙ motifs may have the same amino acid sequence or amino acid sequences different from each other. A plurality of the REPs may have the same amino acid sequence or amino acid sequences different from each other.]

[11-5] The method according to any of [5] to [11], in which the modified fibroin has a limiting oxygen index (LOI) value of 26.0 or more.

[11-6] The method according to any of [5] to [11], in which the modified fibroin has a highest hygroscopic heat generation degree of more than 0.025° C./g, the highest hygroscopic heat generation degree being determined according to Expression A.

highest hygroscopic heat generation degree={(highest temperature of a sample when the sample has been transferred to a high humidity environment after being placed in a low humidity environment until a temperature of the sample reaches equilibrium)−(temperature of the sample when the sample is being transferred to the high humidity environment after being placed in the low humidity environment until the temperature of the sample reaches equilibrium)}(° C.)/sample weight (g) Expression A:

[In Expression A, the low humidity environment means an environment of a temperature of 20° C. and a relative humidity of 40%, and the high humidity environment means an environment of a temperature of 20° C. and a relative humidity of 90%.]

[12] A product including the composite fiber according to any of [1] to [4] and [4-1] to [4-6], in which the product is one selected from the group consisting of fiber, yarn, fabric, knit, braid, non-woven fabric, paper, and cotton.

[13] A side-by-side type composite fiber including a first component and a second component, in which the first component and the second component are joined to each other, and one of the first component and the second component contains a modified fibroin.

[14] A side-by-side type composite fiber including a first component and a second component, in which the first component and the second component are joined to each other, and one of the first component and the second component contains a modified fibroin having a latent crimping ability.

### Advantageous Effects of Invention

According to the present invention, a new fiber having a crimping ability can be provided. According to the present invention, it is possible to provide a composite fiber which is excellent in the crimping ability and is useful as a crimped yarn or a spun yarn. Further, according to the present invention, the composite fiber exhibits high strength and toughness since the composite fiber contains a modified fibroin.

### BRIEF DESCRIPTION OF DRAWINGS

FIG. 1 is a schematic diagram showing a domain sequence of a modified fibroin.

FIG. 2 is a graph showing the distribution of z/w (%) values of a naturally occurring fibroin.

FIG. 3 is a graph showing the distribution of x/y (%) values of a naturally occurring fibroin.

FIG. 4 is a schematic diagram showing a domain sequence of a modified fibroin according to one embodiment.

FIG. 5 is a schematic diagram showing a domain sequence of a modified fibroin according to one embodiment.

FIG. 6 is an illustrative view schematically showing an example of a spinning device for manufacture a composite fiber.

FIG. 7 is a schematic diagram of a composite fiber according to one embodiment of the present invention.

FIG. 8 is a graph showing an example of a result of a hygroscopic heat generating property test.

### DESCRIPTION OF EMBODIMENTS

Hereinafter, embodiments for carrying out the present invention will be described in detail. However, the present invention is not limited to the following embodiments.

A composite fiber according to one embodiment of the present invention includes a first component containing a modified fibroin and a second component containing a structural protein, which are joined to each other in a side-by-side type manner. That is, the cross-sectional shape of the composite fiber according to the present embodiment is a shape in which the first component and the second component are joined to each other, for example, a circular shape in which a semicircle of the first component and a semicircle of the second component are combined. In the cross-sectional shape of the composite fiber, the ratio of the first component to the second component may not be 50:50 but may be properly changed to 30:70, 20:80, or the like. Further, the first component and the second component may be drawn in parallel along the composite fiber or may be twisted spirally. In this case, for example, in one part of the composite fiber, the first component and the second component are arranged one over the other, whereas in the other part of the composite fiber, the first component and the second component are arranged side by side while the joint surface gradually rotates. The hydrophobicity of the modified fibroin and the hydrophobicity of structural protein are different from each other.

The first component of the composite fiber according to the present embodiment contains a modified fibroin. The modified fibroin contained in the first component has a property of shrinking when brought into contact with an aqueous medium described later (hereinafter, also referred to as “water shrinkability”). Further, for example, when a fiber containing the modified fibroin is brought into contact with an aqueous medium in a state where no tension is applied, the fiber is shrunk and crimped. As a result, the latent crimping ability is exhibited in the fiber containing a modified fibroin.

(Modified Fibroin)

The modified fibroin is a protein including a domain sequence represented by Formula 1: [(A)ₙ motif−REP]ₘ. In the modified fibroin, an amino acid sequence (an N-terminal sequence and a C-terminal sequence) may be further added to one or both of the N-terminal side and the C-terminal side of the domain sequence. The N-terminal sequence and the C-terminal sequence, although not limited thereto, are typically regions that do not have repetitions of amino acid motifs characteristic of fibroin and consist of amino acids of about 100 residues. In the present embodiment, in a case where the modified fibroin is a modified spider silk fibroin, the heat retaining property, the hygroscopic heat generating property and/or the flame retardancy are/is more excellent.

The term “modified fibroin” in the present specification means an artificially produced fibroin (an artificial fibroin). The modified fibroin may be a fibroin in which the domain sequence is different from the amino acid sequence of a naturally occurring fibroin or may be the same as the amino acid sequence of a naturally occurring fibroin. The “naturally occurring fibroin” referred to in the present specification is also a protein including a domain sequence represented by Formula 1: [(A)ₙ motif−REP]ₘ.

As the “modified fibroin”, an amino acid sequence of a naturally occurring fibroin may be directly used, a fibroin whose amino acid sequence has been modified based on an amino acid sequence of a naturally occurring fibroin (for example, a fibroin whose amino acid sequence has been modified by modifying a cloned gene sequence of a naturally occurring fibroin) may be used, or a fibroin artificially designed and synthesized independently of a naturally occurring fibroin (for example, a fibroin having a desired amino acid sequence by chemically synthesizing a nucleic acid encoding the designed amino acid sequence) may be used, as long as it has the amino acid sequence specified in the present invention.

The term “domain sequence” as used herein refers to an amino acid sequence which produces a crystalline region (typically, corresponds to (A)ₙ motif of an amino acid sequence) and a non-crystalline region (typically, corresponds to REP of an amino acid sequence) peculiar to fibroin and means an amino acid sequence represented by Formula 1: [(A)ₙ motif−REP]ₘ. Here, the (A)ₙ motif represents an amino acid sequence mainly composed of alanine residues. n may be an integer of 2 to 20, preferably an integer of 4 to 20, more preferably 8 to 20, still more preferably 10 to 20, even still more 4 to 16, even still further preferably 8 to 16, and particularly preferably 10 to 16. In addition, the proportion of the number of alanine residues may be 40% or more, preferably 60% or more, more preferably 70% or more, still more preferably 80% or more, and even still more preferably 90% or more, and even still further preferably 100% (which means that the (A)ₙ motif is composed of only alanine residues), with respect to the total number of amino acid residues in the (A)ₙ motif. The REP indicates an amino acid sequence composed of 2 to 200 amino acid residues and may be an amino acid sequence composed of 10 to 40 amino acid residues, 10 to 60 amino acid residues, 10 to 80 amino acid residues, 10 to 100 amino acid residues, 10 to 120 amino acid residues, 10 to 140 amino acid residues, 10 to 160 amino acid residues, or 10 to 180 amino acids. m represents an integer of 2 to 300 and may be an integer of 8 to 300, 10 to 300, 20 to 300, 40 to 300, 60 to 300, 80 to 300, 10 to 200, 20 to 200, 20 to 180, 20 to 160, 20 to 140, or 20 to 120. A plurality of the (A)ₙ motifs may have the same amino acid sequence or amino acid sequences different from each other. The plurality of the REPs may have the same amino acid sequence or amino acid sequences different from each other. As a specific example of the protein derived from the large spinal canal bookmark silk, a protein including the amino acid sequence set forth in SEQ ID NO: 13 (PRT410) can be mentioned.

The modified fibroin can be obtained, for example, by carrying out the modification of an amino acid sequence equivalent to the substitution, deletion, insertion and/or addition of one or a plurality of amino acid residues with respect to, for example, a cloned gene sequence of a naturally occurring fibroin. The substitution, deletion, insertion, and/or addition of an amino acid residue may be carried out by methods well known to those skilled in the art, such as site-directed mutagenesis. Specifically, the modifications may be carried out by methods described in literature such as Nucleic Acid Res. 10, 6487 (1982) and Methods in Enzymology, 100, 448 (1983).

The naturally occurring fibroin is a protein including a domain sequence represented by Formula 1: [(A)ₙ motif−REP]ₘ, and specifically, for example, a fibroin produced by insects or spiders.

Examples of the fibroin produced by insects include silk proteins produced by silkworms such as Bombyx mori, Bombyx mandarina, Antheraea yamamai, Anteraea pernyi, Eriogyna pyretorum, Pilosamia Cynthia ricini, Samia cynthia, Caligura japonica, Antheraea mylitta, and Antheraea assama; and hornet silk proteins discharged by larvae of Vespa simillima xanthoptera.

More specific examples of the fibroin produced by insects include a silkworm fibroin L chain (GenBank Accession No. M76430 (base sequence) and AAA27840.1 (amino acid sequence)).

Examples of the fibroin produced by spiders include spider silk proteins produced by spiders belonging to the genus Araneus such as Araneus ventricosus, Araneus diadematus, Araneus pinguis, Araneus pentagrammicus and Araneus nojimai, spiders belonging to the genus Neoscona such as Neoscona scylla, Neoscona nautica, Neoscona adianta and Neoscona scylloides, spiders belonging to the genus Pronus such as Pronous minutes, spiders belonging to the genus Cyrtarachne such as Cyrtarachne bufo and Cyrtarachne inaequalis, spiders belonging to the genus Gasteracantha such as Gasteracantha kuhli and Gasteracantha mammosa, spiders belonging to the genus Ordgarius such as Ordgarius hobsoni and Ordgarius sexspinosus, spiders belonging to the genus Argiope such as Argiope amoena, Argiope minuta and Argiope bruennich, spiders belonging to the genus Arachnura such as Arachnura logio, spiders belonging to the genus Acusilas such as Acusilas coccineus, spiders belonging to the genus Cytophora such as Cyrtophora moluccensis, Cyrtophora exanthematica and Cyrtophora unicolor, spiders belonging to the genus Poltys such as Poltys illepidus, spiders belonging to the genus Cyclosa such as Cyclosa octotuberculata, Cyclosa sedeculata, Cyclosa vallata and Cyclosa atrata, and spiders belonging to the genus Chorizopes such as Chorizopes nipponicus; and spider silk proteins produced by spiders belonging to the genus Tetragnatha such as Tetragnatha praedonia, Tetragnatha maxillosa, Tetragnatha extensa and Tetragnatha squamata, spiders belonging to the genus Leucauge such as Leucauge magnifica, Leucauge blanda and Leucauge subblanda, spiders belonging to the genus Nephila such as Nephila clavata and Nephila pilipes, spiders belonging to the genus Menosira such as Menosira ornata, spiders belonging to the genus Dyschiriognatha such as Dyschiriognatha tenera, spiders belonging to the genus Latrodectus such as Latrodectus mactans, Latrodectus hasseltii, Latrodectus geometricus and Latrodectus tredecimguttatus, and spiders belonging to the family Tetragnathidae such as spiders belonging to the genus Euprosthenops. Examples of spider silk proteins include traction yarn proteins such as MaSp (MaSp1 and MaSp2) and ADF (ADF3 and ADF4), and MiSp (MiSp1 and MiSp2).

More specific examples of the fibroin produced by spiders include fibroin-3 (adf-3) [derived from Araneus diadematus] (GenBank Accession No. AAC47010 (amino acid sequence), U47855 (base sequence)), fibroin-4 (adf-4) [derived from Araneus diadematus] (GenBank Accession No. AAC47011 (amino acid sequence), U47856 (base sequence)), dragline silk protein spidroin 1 [derived from Nephila clavipes] (GenBank Accession No. AAC04504 (amino acid sequence), U37520 (base sequence)), major ampullate spidroin 1 [derived from Latrodectus hesperus] (GenBank Accession No. ABR68856 (amino acid sequence), EF595246 (base sequence)), dragline silk protein spidroin 2 [derived from Nephila clavata] (GenBank Accession No. AAL32472 (amino acid sequence), AF441245 (base sequence)), major ampullate spidroin 1 [derived from Euprosthenops australis] (GenBank Accession No. CAJ00428 (amino acid sequence), AJ973155 (base sequence)) and major ampullate spidroin 2 [Euprosthenops australis] (GenBank Accession No. CAM32249.1 (amino acid sequence), AM490169 (base sequence)), minor ampullate silk protein 1 [Nephila clavipes] (GenBank Accession No. AAC14589.1 (amino acid sequence), minor ampullate silk protein 2 [Nephila clavipes] (GenBank Accession No. AAC14591.1 (amino acid sequence)), and minor ampullate spidroin-like protein [Nephilengys cruentata] (GenBank Accession No. ABR37278.1 (amino acid sequence)).

As a further specified example of the naturally occurring fibroin, a fibroin whose sequence information is registered in NCBI GenBank may be mentioned. For example, sequences thereof may be confirmed by extracting sequences in which spidroin, ampullate, fibroin, “silk and polypeptide”, or “silk and protein” is described as a keyword in DEFINITION among sequences including INV as DIVISION in sequence information registered in NCBI GenBank, sequences in which a specific character string of a product is described from CDS or sequences in which a specific character string is described from SOURCE to TISSUE TYPE.

The modified fibroin may be a modified silk fibroin (a modified silk protein obtained by modifying an amino acid sequence of a silk protein produced by silkworm), and a modified spider silk fibroin (a modified spider silk protein obtained by modifying an amino acid sequence of a spider silk protein produced by spiders). The modified fibroin is preferably a modified spider silk fibroin.

Specific examples of the modified fibroin include: a modified fibroin (first modified fibroin) derived from a large spinal canal bookmark silk protein produced in a major ampullate gland of a spider; a modified fibroin (second modified fibroin) in which the content of the glycine residue is reduced; a modified fibroin (third modified fibroin) in which the content of the (A)ₙ motif is reduced; and a modified fibroin (fourth modified fibroin) in which a content of glycine residue and the content of the (A)ₙ motif are reduced. These modified fibroins are excellent in flame retardancy, hygroscopic heat generating property, and heat retaining property and suitable for using for fireproof clothes (for example, for fireman uniforms and for rescue), fireproof gloves (for example, for laboratory, for industries, and for cooking), winter clothes (cold protection clothes) such as gloves, mufflers, sweaters, outerwear and jackets, batting for cold protection clothes, innerwear, sportswear, shirts, bedding, and batting for bedding.

The first modified fibroin include a protein including a domain sequence represented by Formula 1: [(A)ₙ motif−REP]ₘ. In the first modified fibroin, the number of amino acid residues in the (A)ₙ motif is preferably an integer of 3 to 20, more preferably an integer of 4 to 20, still more preferably an integer of 8 to 20, even more preferably an integer of 10 to 20, even further more preferably an integer of 4 to 16, particularly preferably an integer of 8 to 16, and most preferably an integer of 10 to 16. In the first modified fibroin, the number of amino acid residues constituting REP in Formula 1 is preferably 10 to 200 residues, more preferably 10 to 150 residues, and still more preferably 20 to 100 residues, and even more preferably 20 to 75 residues. In the first modified fibroin, the total number of glycine residues, serine residues, and alanine residues contained in the amino acid sequence represented by Formula 1: [(A)ₙ motif−REP]ₘ is preferably 40% or more, more preferably 60% or more, and still more preferably 70% or more with respect to the total number of amino acid residues.

The first modified fibroin may be a polypeptide including an amino acid sequence unit represented by Formula 1: [(A)ₙ motif−REP]ₘ, and having a C-terminal sequence which is the amino acid sequence set forth in any of SEQ ID NOs: 1 to 3 or an amino acid sequence having 90% or more homology with the amino acid sequence set forth in any of SEQ ID NOs: 1 to 3.

The amino acid sequence set forth in SEQ ID NO: 1 is identical to the amino acid sequence consisting of 50 amino acid residues at the C-terminal of the amino acid sequence of ADF3 (GI: 1263287, NCBI). The amino acid sequence set forth in SEQ ID NO: 2 is identical to the amino acid sequence obtained by removing 20 residues from the C-terminal of the amino acid sequence set forth in SEQ ID NO: 1. The amino acid sequence set forth in SEQ ID NO: 3 is identical to the amino acid sequence obtained by removing 29 residues from the C-terminal of the amino acid sequence set forth in SEQ ID NO: 1.

More specific examples of the first modified fibroin can include a modified fibroin including (1-i) the amino acid sequence set forth in SEQ ID NO: 4 (recombinant spider silk protein ADF3KaiLargeNRSH1) and (1-ii) an amino acid sequence having 90% or more sequence identity with the amino acid sequence set forth in SEQ ID NO: 4. The sequence identity is preferably 95% or more.

The amino acid sequence set forth in SEQ ID NO: 4 is an amino acid sequence obtained by approximately doubling repeating regions from the first repeating region to the 13th repeating region and performing mutation so that translation is terminated at the 1154th amino acid residue in an amino acid sequence obtained by adding the amino acid sequence (SEQ ID NO: 5) consisting of a start codon, a His10 tag, and a recognition site for HRV3C protease (human rhinovirus 3C protease) to the N-terminal of ADF3. The C-terminal amino acid sequence of the amino acid sequence set forth in SEQ ID NO: 4 is identical to the amino acid sequence set forth in SEQ ID NO: 3.

The modified fibroin of (1-i) may consist of the amino acid sequence set forth in SEQ ID NO: 4.

The domain sequence of the second modified fibroin has an amino acid sequence in which the content of the glycine residue is reduced, as compared with naturally occurring fibroin. It can be said that the second modified fibroin has an amino acid sequence equivalent to an amino acid sequence in which at least one or a plurality of glycine residues in the REP are substituted with other amino acid residues, as compared with naturally occurring fibroin.

The domain sequence of the second modified fibroin may have an amino acid sequence equivalent to an amino acid sequence in which one glycine residue in at least one or the plurality of motif sequences, at least one of which is selected from GGX and GPGXX (where G represents a glycine residue, P represents a proline residue, and X represents any amino acid residue other than glycine) in the REP, is substituted with other amino acid residue, as compared with naturally occurring fibroin.

In the second modified fibroin, the proportion of the motif sequences in which the above-described glycine residue is substituted with other amino acid residue may be 10% or more with respect to the entire motif sequences.

The second modified fibroin may include a domain sequence represented by Formula 1: [(A)ₙ motif−REP]ₘ and have an amino acid sequence in which z/w is 30% or more, 40% or more, 50% or more, or 50.9% or more, in a case where the total number of amino acid residues in the amino acid sequence consisting of XGX (where X represents any amino acid residue other than glycine) included in all REPs in a sequence excluding the sequence from the (A)ₙ motif located at the most C-terminal side to the C-terminal of the domain sequence from the domain sequence is denoted by z, and the total number of amino acid residues in the sequence excluding the sequence from the (A)ₙ motif located at the most C-terminal side to the C-terminal of the domain sequence from the domain sequence is denoted by w. The number of alanine residues may be 83% or more with respect to the total number of amino acid residues in the (A)ₙ motif, preferably 86% or more, more preferably 90% or more, still more preferably 95% or more, and even still more preferably 100% (which means that the (A)ₙ motif is composed of only alanine residues).

In the second modified fibroin, the content proportion of an amino acid sequence consisting of XGX is preferably increased by substituting one glycine residue in GGX motif with other amino acid residue. In the second modified fibroin, the content proportion of an amino acid sequence consisting of GGX in the domain sequence is preferably 30% or less, more preferably 20% or less, still more preferably 10% or less, even still more preferably 6% or less, still further preferably 4% or less, and particularly preferably 2% or less. The content proportion of an amino acid sequence consisting of GGX in a domain sequence can be calculated by the same method as the following method for calculating the content proportion (z/w) of the amino acid sequence consisting of XGX.

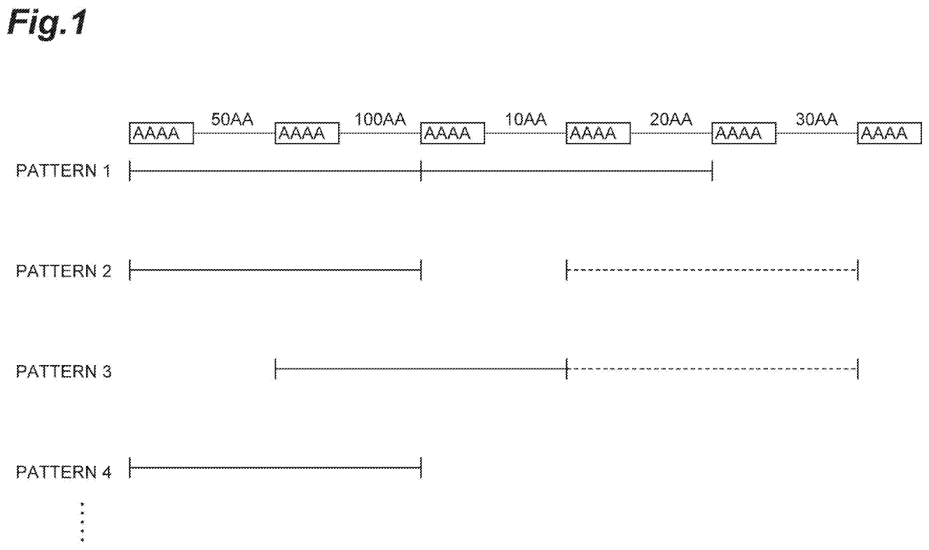

The calculation method for z/w will be described in more detail. First, in a fibroin (a modified fibroin or a naturally occurring fibroin) including a domain sequence represented by Formula 1: ([(A)ₙ motif−REP]ₘ−(A)ₙ motif], the amino acid sequence consisting of XGX is extracted from all REPs included in a sequence excluding a sequence from the (A)ₙ motif located closest to the C-terminal side to the C-terminal of the domain sequence from the domain sequence. The total number of amino acid residues constituting XGX is z. For example, in a case where 50 amino acid sequences consisting of XGX (without overlap) are extracted, z is 50×3=150. Further, for example, in a case where there exists an X (a central X) contained in two XGXs, as in the case of an amino acid sequence consisting of XGXGX, the calculation is performed by subtracting the overlapping portion (in the case of XGXGX, it is counted as 5 amino acid residues). w is the total number of amino acid residues included in the sequence excluding a sequence from the (A)ₙ motif located closest to the C terminal to the C terminal of the domain sequence from the domain sequence. For example, in the case of the domain sequence illustrated in FIG. 1, w is 4+50+4+100+4+10+4+20+4+30=230 (the (A)ₙ motif located closest to the C-terminal side is excluded.). Next, z/w (%) can be calculated by dividing z by w.

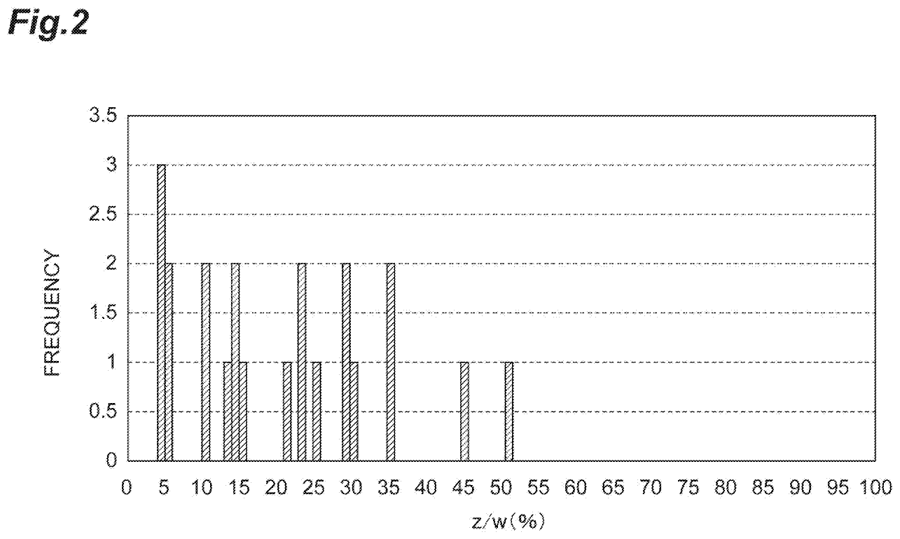

Here, z/w in a naturally occurring fibroin will be described. First, 663 types of fibroins (among them, 415 types of fibroins derived from spiders) were extracted by confirming fibroins with amino acid sequence information registered in NCBI GenBank according to the method exemplified as described above. By the calculation method described above, z/w was calculated from the amino acid sequence of each naturally occurring fibroin, among all of the extracted fibroins, which includes the domain sequence represented by Formula 1: [(A)ₙ motif−REP]ₘ and in which a content proportion of the amino acid sequence consisting of GGX in fibroin is 6% or less. The results are shown in FIG. 2. The horizontal axis in FIG. 2 indicates z/w (%), and the vertical axis indicates frequency. As is clear from FIG. 2, z/w in the naturally occurring fibroin is less than 50.9% (the highest z/w is 50.86%).

In the second modified fibroin, z/w is preferably 50.9% or more, more preferably 56.1% or more, still more preferably 58.7% or more, even still more preferably 70% or more, and still further preferably 80% or more. The upper limit of z/w is not particularly limited, but, for example, it may be 95% or less.

The second modified fibroin can be obtained by, for example, modifying a cloned naturally occurring fibroin gene sequence such that at least a part of a base sequence encoding a glycine residue is substituted with other amino acid residue to encode other amino acid residue. In this case, one glycine residue in GGX motif and GPGXX motif may be selected as the glycine residue to be modified or may be substituted so that z/w is 50.9% or more. Alternatively, a modified fibroin may also be obtained, for example, by designing an amino acid sequence satisfying the above-described aspect based on the amino acid sequence of a naturally occurring fibroin and chemically synthesizing a nucleic acid encoding the designed amino acid sequence. In any case, with respect to the amino acid sequence of a naturally occurring fibroin, in addition to the modification corresponding to the substitution of the glycine residue in the REP with other amino acid residue, further modification of amino acid sequence corresponding to substitution, deletion, insertion and/or addition of one or a plurality of amino acid residues may be carried out.

The other amino acid residue described above is not particularly limited as long as it is any amino acid residue other than glycine residue, but it is preferably a hydrophobic amino acid residue such as valine (V) residue, leucine (L) residue, isoleucine (I) residue, methionine (M) residue, proline (P) residue, phenylalanine (F) residue, and tryptophan (W) residue, or a hydrophilic amino acid residues such glutamine (Q) residue, asparagine (N) residue, serine (S) residue, lysine (K) residue, and glutamic acid (E) residue, more preferably valine (V) residue, phenylalanine (F), leucine (L) residue, isoleucine (I) residue, and glutamine (Q) residue, and still more preferably glutamine (Q) residue.

A more specific example of the fibroin can include a modified fibroin including (2-i) the amino acid sequence set forth in SEQ ID NO: 6 (Met-PRT380), SEQ ID NO: 7 (Met-PRT410), SEQ ID NO: 8 (Met-PRT525), or SEQ ID NO: 9 (Met-PRT799), or (2-ii) an amino acid sequence having 90% or more sequence identity with the amino acid sequence set forth in SEQ ID NO: 6, SEQ ID NO: 7, SEQ ID NO: 8, or SEQ ID NO: 9.

The modified fibroin of (2-i) will be described. The amino acid sequence set forth in SEQ ID NO: 6 is obtained by substituting all GGXs in the REP of the amino acid sequence set forth in SEQ ID NO: 10 (Met-PRT313) equivalent to a naturally occurring fibroin with GQX. The amino acid sequence set forth in SEQ ID NO: 7 is obtained by deleting one of every two (A)ₙ motifs from the N-terminal side to the C-terminal side in the amino acid sequence set forth in SEQ ID NO: 6 and further inserting one [(A)ₙ motif−REP] just before the C-terminal sequence. The amino acid sequence set forth in SEQ ID NO: 8 is obtained by inserting two alanine residues at the C-terminal side of each (A)ₙ motif of the amino acid sequence set forth in SEQ ID NO: 7, and further substituting a part of glutamine (Q) residues with serine (S) residues and deleting a part of amino acids on the C-terminal side. The amino acid sequence set forth in SEQ ID NO: 9 is an amino acid sequence obtained by adding a Hinge and a His tag to the C-terminal of a sequence obtained by repeating, four times, a region of 20 domain sequences (where several amino acid residues on the C-terminal side of the region are substituted) present in the amino acid sequence set forth in SEQ ID NO: 7.

The value of z/w in the amino acid sequence set forth SEQ ID NO: 10 (equivalent to a naturally occurring fibroin) is 46.8%. The values of z/w in the amino acid sequences set forth in SEQ ID NO: 6, the amino acid sequence set forth in SEQ ID NO: 7, the amino acid sequence set forth in SEQ ID NO: 8, and the amino acid sequence set forth in SEQ ID NO: 9 are respectively 58.7%, 70.1%, 66.1%, and 70.0%. In addition, the values of x/y with a Giza ratio (described later) of 1:1.8 to 11.3 in the amino acid sequences set forth in SEQ ID NO: 10, SEQ ID NO: 6, SEQ ID NO: 7, SEQ ID NO: 8, and SEQ ID NO: 9 are respectively 15.0%, 15.0%, 93.4%, 92.7%, and 89.8%.

The modified fibroin of (2-i) may consist of the amino acid sequence set forth in SEQ ID NO: 6, SEQ ID NO: 7, SEQ ID NO: 8, or SEQ ID NO: 9.

The modified fibroin of (2-ii) includes an amino acid sequence having 90% or more sequence identity with the amino acid sequence set forth in SEQ ID NO: 6, SEQ ID NO: 7, SEQ ID NO: 8, or SEQ ID NO: 9. The modified fibroin of (2-ii) is also a protein including a domain sequence represented by Formula 1: [(A)ₙ motif−REP]ₘ. The sequence identity is preferably 95% or more.

The modified fibroin of (2-ii) preferably has 90% or more sequence identity with the amino acid sequence set forth in SEQ ID NO: 6, SEQ ID NO: 7, SEQ ID NO: 8, or SEQ ID NO: 9, and in a case where the total number of amino acid residues in the amino acid sequence consisting of XGX (where X represents any amino acid residue other than glycine) included in the REP is z, and the total number of amino acid residues in the REP in the domain sequence is w, z/w is preferably 50.9% or more.

The second modified fibroin may include a tag sequence at one or both of the N-terminal and C-terminal. This makes it possible to isolate, immobilize, detect, and visualize the modified fibroin.

The tag sequence may be, for example, an affinity tag utilizing specific affinity (binding property, affinity) with another molecule. As a specific example of the affinity tag, a histidine tag (a His tag) can be mentioned. The His tag is a short peptide in which about 4 to 10 histidine residues are arranged and has a property of specifically binding to a metal ion such as nickel, and thus it can be used for isolation of a modified fibroin by a chelating metal chromatography. A specific example of the tag sequence may include the amino acid sequence set forth in SEQ ID NO: 11 (amino acid sequence including a His tag sequence and a hinge sequence).

In addition, a tag sequence such as glutathione-S-transferase (GST) that specifically binds to glutathione or a maltose binding protein (MBP) that specifically binds to maltose can also be used.

Further, an “epitope tag” utilizing an antigen-antibody reaction can also be used. By adding a peptide (an epitope) showing antigenicity as a tag sequence, an antibody against the epitope can be bound. Examples of the epitope tag include an HA (peptide sequence of hemagglutinin of influenza virus) tag, a myc tag, and a FLAG tag. The modified fibroin can be easily purified with high specificity by utilizing an epitope tag.

It is also possible to use a tag sequence which can be cleaved with a specific protease. By treating a protein adsorbed via the tag sequence with a protease, it is also possible to recover a modified fibroin cleaved from the tag sequence.

A more specific example of the modified fibroin having a tag sequence can include a modified fibroin including (2-iii) the amino acid sequence set forth in SEQ ID NO: 12 (Met-PRT380), SEQ ID NO: 13 (Met-PRT410), SEQ ID NO: 14 (Met-PRT525), or SEQ ID NO: 15 (Met-PRT799), or (2-iv) an amino acid sequence having 90% or more sequence identity with the amino acid sequence set forth in SEQ ID NO: 12, SEQ ID NO: 13, SEQ ID NO: 14, or SEQ ID NO: 15.

The amino acid sequences set forth in SEQ ID NO: 16 (PRT313), SEQ ID NO: 12, SEQ ID NO: 13, SEQ ID NO: 14, and SEQ ID NO: 15 are respectively amino acid sequences obtained by adding the amino acid sequence (including a His tag sequence and a hinge sequence) set forth in SEQ ID NO: 11 to the N-terminal of the amino acid sequences set forth in SEQ ID NO: 10, SEQ ID NO: 6, SEQ ID NO: 7, SEQ ID NO: 8, and SEQ ID NO: 9.

The modified fibroin of (2-iii) may consist of the amino acid sequence set forth in SEQ ID NO: 12, SEQ ID NO: 13, SEQ ID NO: 14, or SEQ ID NO: 15.

The modified fibroin of 2-iv) includes an amino acid sequence having 90% or more sequence identity with the amino acid sequence set forth in SEQ ID NO: 12, SEQ ID NO: 13, SEQ ID NO: 14, or SEQ ID NO: 15. The modified fibroin of (2-iv) is also a protein including a domain sequence represented by Formula 1: [(A)ₙ motif−REP]ₘ. The sequence identity is preferably 95% or more.

The modified fibroin of (2-iv) preferably has 90% or more sequence identity with the amino acid sequence set forth in SEQ ID NO: 12, SEQ ID NO: 13, SEQ ID NO: 14, or SEQ ID NO: 15, and in a case where the total number of amino acid residues in the amino acid sequence consisting of XGX (where X represents any amino acid residue other than glycine) included in the REP is z, and the total number of amino acid residues in the REP in the domain sequence is w, z/w is preferably 50.9% or more.

The second modified fibroin may include a secretory signal for releasing the protein produced in the recombinant protein production system to the outside of a host. The sequence of the secretory signal can be appropriately set depending on the type of the host.

The domain sequence of the third modified fibroin has an amino acid sequence in which the content of the (A)ₙ motif is reduced, as compared with naturally occurring fibroin. It can be said that the domain sequence of the third modified fibroin has an amino acid sequence equivalent to an amino acid sequence in which at least one or a plurality of the (A)ₙ motifs are deleted, as compared with naturally occurring fibroin.

The third modified fibroin may have an amino acid sequence equivalent to an amino acid sequence in which 10% to 40% of the (A)ₙ motifs are deleted from naturally occurring fibroin.

The domain sequence of the third modified fibroin may have an amino acid sequence equivalent to an amino acid sequence obtained by deleting at least one of every one to three (A)ₙ motifs from the N-terminal side to the C-terminal side, as compared with naturally occurring fibroin.

The domain sequence of the third modified fibroin may have an amino acid sequence equivalent to an amino acid sequence obtained by repeating deletion of at least two consecutive (A)ₙ motifs and deletion of one (A)ₙ motif in this order from the N-terminal side to the C-terminal side, as compared with naturally occurring fibroin.

The domain sequence of the third modified fibroin may have an amino acid sequence equivalent to an amino acid sequence obtained by deleting at least one of every two (A)ₙ motifs from the N-terminal side to the C-terminal side.

The third modified fibroin may include a domain sequence represented by Formula 1: [(A)ₙ motif−REP]ᵢₙ, and may have an amino acid sequence in which x/y is 20% or more, 30% or more, 40% or more, or 50% or more, in a case where the number of amino acid residues of the REP of two [(A)ₙ motif−REP] units adjacent to each other is sequentially compared from the N-terminal side to the C-terminal side and then the number of amino acid residues of one REP having a small number of amino acid residues is set to 1, the maximum total value of the added number of amino acid residues of two [(A)ₙ motif−REP] units adjacent to each other, in which the ratio of the number of amino acid residues of the other REP is 1.8 to 11.3, is denoted by x, and the total number of amino acid residues in the domain sequence is denoted by y. The number of alanine residues may be 83% or more with respect to the total number of amino acid residues in the (A)ₙ motif, preferably 86% or more, more preferably 90% or more, still more preferably 95% or more, and even still more preferably 100% (which means that the (A)ₙ motif is composed of only alanine residues).

The method for calculating x/y will be described in more detail with reference to FIG. 1. FIG. 1 illustrates a domain sequence obtained by removing an N-terminal sequence and a C-terminal sequence from a modified fibroin. The domain sequence has a sequence of, from the N-terminal side (left side), (A)ₙ motif-first REP (50 amino acid residues)-(A)ₙ motif-second REP (100 amino acid residues)-(A)ₙ motif-third REP (10 amino acid residues)-(A)ₙ motif-fourth REP (20 amino acid residues)-(A)ₙ motif-fifth REP (30 amino acid residues)-(A)ₙ motif sequence.

Two [(A)ₙ motif−REP] units adjacent to each other are sequentially selected from the N-terminal side toward the C-terminal side so that the units are not overlapped with each other. In this case, an unselected [(A)ₙ motif−REP] unit may be present. In FIG. 1, pattern 1 (comparison of first REP and second REP, and comparison of third REP and fourth REP), pattern 2 (comparison of first REP and second REP, and comparison of fourth REP and fifth REP), pattern 3 (comparison of second REP and third REP, and comparison of fourth REP and fifth REP), and pattern 4 (comparison of first REP and second REP). There are other selection methods other than these methods.

Subsequently, for each pattern, the number of amino acid residues of each REP in two selected [(A)ₙ motif−REP] units adjacent to each other is compared. The comparison is performed by determining the ratio of the number of amino acid residues of one REP to the number of amino acid residues of the other REP in a case where the smaller number of amino acid residues of the REP is set to 1. For example, in a case of comparing the first REP (50 amino acid residues) and the second REP (100 amino acid residues), in a case where the first REP having the smaller number of amino acid residues is set to 1, the ratio of the number of amino acid residues of the second REP is 100/50=2. Similarly, in a case of comparing the fourth REP (20 amino acid residues) and the fifth REP (30 amino acid residues), in a case where the fourth REP having the smaller number of amino acid residues is set to 1, the ratio of the number of amino acid residues of the fifth REP is 30/20=1.5.

In FIG. 1, in a case where one group of [(A)ₙ motif−REP] units having the smaller number of amino acid residues is set to 1, the other group in which the ratio of the number of amino acid residues is 1.8 to 11.3 is indicated by a solid line. In the present specification, this ratio is referred to as the Giza ratio. In a case where one group of [(A)ₙ motif−REP] units having the smaller number of amino acid residues is set to 1, the other group in which the ratio of the number of amino acid residues is less than 1.8 or more than 11.3 is indicated by a broken line.

In each pattern, the total numbers of amino acid residues of two [(A)ₙ motif−REP] units adjacent to each other indicated by solid lines are added (not only the number of the REPs but also the number of amino acid residues in the (A)ₙ motif are added.) Then, the added total values are compared, and the total value (maximum value of the total values) of the pattern having the maximum total value is denoted by x. In the example illustrated in FIG. 1, the total value of the pattern 1 is the maximum.

Next, x/y (%) can be calculated by dividing x by y which is the total number of amino acid residues of the domain sequence.

In the third modified fibroin, x/y is preferably 50% or more, more preferably 60% or more, still more preferably 65% or more, even still more preferably 70% or more, still further preferably 75% or more, and particularly preferably 80% or more. The upper limit of x/y is not particularly limited, but for example, it may be 100% or less. In a case where the Giza ratio is 1:1.9 to 11.3, x/y is preferably 89.6% or more. In a case where the Giza ratio is 1:1.8 to 3.4, x/y is more preferably 77.1% or more. In a case where the Giza ratio is 1:1.9 to 8.4, x/y is still more preferably 75.9% or more. In a case where the Giza ratio is 1:1.9 to 4.1, x/y is even still more preferably 64.2% or more.

In a case where the third modified fibroin is a modified fibroin in which at least seven (A)ₙ motifs present in the domain sequence are composed of only alanine residues, x/y is preferably 46.4% or more, more preferably 50% or more, still more preferably 55% or more, even still more preferably 60% or more, still further preferably 70% or more, and particularly preferably 80% or more. The upper limit of x/y is not particularly limited as long as it is 100% or less.

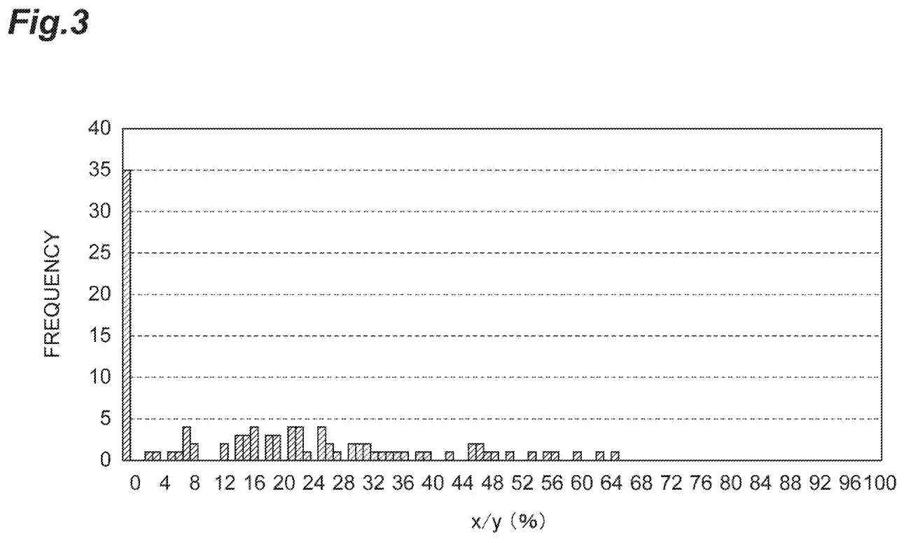

Here, x/y in a naturally occurring fibroin will be described. First, 663 types of fibroins (among them, 415 types of fibroins derived from spiders) were extracted by confirming fibroins with amino acid sequence information registered in NCBI GenBank according to the method exemplified as described above. By the calculation method described above, x/y was calculated from the amino acid sequence of each naturally occurring fibroin, among all of the extracted fibroins, composed of the domain sequence represented by Formula 1: [(A)ₙ motif−REP]ₘ. The results in the case where the Giza ratio is 1:1.9 to 4.1 are shown in FIG. 3.

The horizontal axis in FIG. 3 indicates x/y (%), and the vertical axis indicates frequency. As is clear from FIG. 3, x/y in the naturally occurring fibroin is less than 64.2% (the highest x/y is 64.14%).

The third modified fibroin, for example, can be obtained by deleting one or a plurality sequences encoding (A)ₙ motif from a cloned gene sequence of naturally occurring fibroin such that x/y is 64.2% or more. Alternatively, the third modified fibroin may also be obtained, for example, by designing an amino acid sequence equivalent to an amino acid sequence obtained by deleting one or a plurality (A)ₙ motifs so that x/y is 64.2% or more based on the amino acid sequence of a naturally occurring fibroin and chemically synthesizing a nucleic acid encoding the designed amino acid sequence. In any case, with respect to the amino acid sequence of a naturally occurring fibroin, in addition to the modification corresponding to the deletion of the (A)ₙ motif, further modification of amino acid sequence equivalent to substitution, deletion, insertion and/or addition of one or a plurality of amino acid residues may be carried out.

A more specific example of the third modified fibroin can include a modified fibroin including (3-i) the amino acid sequence set forth in SEQ ID NO: 17 (Met-PRT399), SEQ ID NO: 7 (Met-PRT410), SEQ ID NO: 8 (Met-PRT525), or SEQ ID NO: 9 (Met-PRT799), or (3-ii) an amino acid sequence having 90% or more sequence identity with the amino acid sequence set forth in SEQ ID NO: 17, SEQ ID NO: 7, SEQ ID NO: 8, or SEQ ID NO: 9.

The modified fibroin of (3-i) will be described. The amino acid sequence set forth in SEQ ID NO: 17 is obtained by deleting one of every two (A)ₙ motifs from the N-terminal side to the C-terminal side in the amino acid sequence set forth in SEQ ID NO: 10 (Met-PRT313) equivalent to a naturally occurring fibroin and by further inserting one [(A)ₙ motif−REP] just before the C-terminal sequence. The amino acid sequence set forth in SEQ ID NO: 7, SEQ ID NO: 8, or SEQ ID NO: 9 is as described in the second modified fibroin.

The value of x/y with a Giza ratio of 1:1.8 to 11.3 in the amino acid sequence set forth in SEQ ID NO: 10 (equivalent to a naturally occurring fibroin) is 15.0%. Both the values of x/y in the amino acid sequences set forth in SEQ ID NO: 17 and the value of x/y in the amino acid sequence set forth in SEQ ID NO: 7 are 93.4%. The value of x/y in the amino acid sequence set forth in SEQ ID NO: 8 is 92.7%. The value of x/y in the amino acid sequence set forth in SEQ ID NO: 9 is 89.8%. The values of z/w in the amino acid sequences set forth in SEQ ID NO: 10, SEQ ID NO: 17, SEQ ID NO: 7, SEQ ID NO: 8, and SEQ ID NO: 9 are respectively 46.8%, 56.2%, 70.1%, 66.1%, and 70.0%.

The modified fibroin of (3-i) may consist of the amino acid sequence set forth in SEQ ID NO: 17, SEQ ID NO: 7, SEQ ID NO: 8, or SEQ ID NO: 9.

The modified fibroin of (3-ii) includes an amino acid sequence having 90% or more sequence identity with the amino acid sequence set forth in SEQ ID NO: 17, SEQ ID NO: 7, SEQ ID NO: 8, or SEQ ID NO: 9. The modified fibroin of (3-ii) is also a protein including a domain sequence represented by Formula 1: [(A)ₙ motif−REP]ₘ. The sequence identity is preferably 95% or more.

The modified fibroin of (3-ii) preferably has 90% or more sequence identity with the amino acid sequence set forth in SEQ ID NO: 17, SEQ ID NO: 7, SEQ ID NO: 8, or SEQ ID NO: 9, and in a case where the number of amino acid residues of the REP of two [(A)ₙ motif−REP] units adjacent to each other is sequentially compared from the N-terminal side to the C-terminal side, then the number of amino acid residues of one REP having a small number of amino acid residues is set to 1, and the maximum total value of the added numbers of amino acid residues of two [(A)ₙ motif−REP] units adjacent to each other, in which the ratio (1:1.8 to 11.3 as a Giza ratio) of the number of amino acid residues of the other REP is 1.8 to 11.3, is denoted by x, and the total number of amino acid residues in the domain sequence is denoted by y, x/y is preferably 64.2% or more.

The third modified fibroin may include a tag sequence described above at one or both of the N-terminal and C-terminal.

A more specific example of the modified fibroin having a tag sequence can includes a modified fibroin including (3-iii) the amino acid sequence set forth in SEQ ID NO: 18 (Met-PRT399), SEQ ID NO: 13 (Met-PRT410), SEQ ID NO: 14 (Met-PRT525), or SEQ ID NO: 15 (Met-PRT799), or (3-iv) an amino acid sequence having 90% or more sequence identity with the amino acid sequence set forth in SEQ ID NO: 18, SEQ ID NO: 13, SEQ ID NO: 14, or SEQ ID NO: 15.

The amino acid sequences set forth in SEQ ID NO: 18, SEQ ID NO: 13, SEQ ID NO: 14, and SEQ ID NO: 15 are respectively amino acid sequences obtained by adding the amino acid sequence (including a His tag sequence and a hinge sequence) set forth in SEQ ID NO: 11 to the N-terminal of the amino acid sequences set forth in SEQ ID NO: 17, SEQ ID NO: 7, SEQ ID NO: 8, and SEQ ID NO: 9.

The modified fibroin of (3-iii) may consist of the amino acid sequence set forth in SEQ ID NO: 18, SEQ ID NO: 13, SEQ ID NO: 14, or SEQ ID NO: 15.

The modified fibroin of (3-iv) includes an amino acid sequence having 90% or more sequence identity with the amino acid sequence set forth in SEQ ID NO: 18, SEQ ID NO: 13, SEQ ID NO: 14, or SEQ ID NO: 15. The modified fibroin of (3-iv) is also a protein including a domain sequence represented by Formula 1: [(A)ₙ motif−REP]ₘ. The sequence identity is preferably 95% or more.

The modified fibroin of (3-iv) preferably has 90% or more sequence identity with the amino acid sequence set forth in SEQ ID NO: 18, SEQ ID NO: 13, SEQ ID NO: 14, or SEQ ID NO: 15, and in a case where the number of amino acid residues of the REP of two [(A)ₙ motif−REP] units adjacent to each other is sequentially compared from the N-terminal side to the C-terminal side, then the number of amino acid residues of one REP having a small number of amino acid residues is set to 1, the maximum total value of the added numbers of amino acid residues of two [(A)ₙ motif−REP] units adjacent to each other, in which the ratio of the number of amino acid residues of the other REP is 1.8 to 11.3, is denoted by x, and the total number of amino acid residues in the domain sequence is denoted by y, x/y is preferably 64.2% or more.

The third modified fibroin may include a secretory signal for releasing the protein produced in the recombinant protein production system to the outside of a host. The sequence of the secretory signal can be appropriately set depending on the type of the host.

The domain sequence of the fourth modified fibroin has an amino acid sequence having not only a reduced content of the (A)ₙ motif but also a reduced content of the glycine residue, as compared with naturally occurring fibroin. It can be said that the domain sequence of the fourth modified fibroin has an amino acid sequence equivalent to an amino acid sequence in which at least one or a plurality of the (A)ₙ motifs are deleted and at least one or a plurality of glycine residues in the REP are further substituted with other amino acid residues, as compared with naturally occurring fibroin. That is, the fourth modified fibroin is a modified fibroin having the characteristics of the second modified fibroin and the third modified fibroin described above. Specific aspects and the like of the fourth modified fibroin are as described in the second modified fibroin and the third modified fibroin.

A more specific example of the fourth modified fibroin can include a modified fibroin including (4-i) the amino acid sequence set forth in SEQ ID NO: 7 (Met-PRT410), SEQ ID NO: 8 (Met-PRT525), or SEQ ID NO: 9 (Met-PRT799), SEQ ID NO: 13 (PRT410), SEQ ID NO: 14 (PRT525), or SEQ ID NO: 15 (PRT799), or (4-ii) an amino acid sequence having 90% or more sequence identity with the amino acid sequence set forth in SEQ ID NO: 7, SEQ ID NO: 8, SEQ ID NO: 9, SEQ ID NO: 13, SEQ ID NO: 14, or SEQ ID NO: 15. Specific aspects of the modified fibroin including the amino acid sequence set forth SEQ ID NO: 7, SEQ ID NO: 8, SEQ ID NO: 9, SEQ ID NO: 13, SEQ ID NO: 14, or SEQ ID NO: 15 are as described above.

The structural protein contained in the second component may be a synthetic fiber or a hydrophobic modified fibroin.

The synthetic fiber is generally hard to be wetted by water and has poor water absorbency and poor hygroscopicity. Examples of the synthetic fiber include polyesters such as polyethylene terephthalate, polyamides such as a polycaproamide (nylon 6) and nylon 66, polyacryl, and polyvinyl formal (vinylon). The synthetic fiber generally has the property of not easily being shrunk even when brought into contact with an aqueous medium.

The second component may be a chemical fiber. Examples of the chemical fibers include a natural polymer (a natural fiber), a semi-synthetic polymer (a semi-synthetic fiber), and a synthetic polymer (a synthetic fiber). Examples of the natural polymer (the natural fiber) include regenerated cellulose fibers such as rayon, cupra, polynosic, and lyocell.

Examples of the semi-synthetic polymer (semi-synthetic fiber) include acetate fibers such as an acetate (diacetate) fiber and a triacetate fiber, and PROMIX.

Examples of the synthetic polymer (synthetic fiber) include the above-described polyesters such as polyethylene terephthalate, polyamides such as a polycaproamide (nylon 6) and nylon 66, polyacryl, polyvinyl formal (vinylon), and polyurethane (spandex).

In a case where a regenerated cellulose fiber, an acetate fiber, PROMIX, polyacryl, polyvinyl formal, polyurethane, or the like is used as the second component, it is dissolved in a known solvent (solution) to prepare a spinning solution (doping liquid), which is discharged from the spinneret and joined to the first component (modified fibroin). Then, the first component (modified fibroin) and the second component (chemical fiber) are solidified in a coagulation liquid, whereby a side-by-side type composite fiber is obtained.

In a case where polyester or polyamide is used as the second component, a raw material thereof is melted to form a liquid, which is discharged from the spinneret and joined to the first component (modified fibroin). Then, the first component (modified fiboroin) and the second component (chemical fiber) are solidified in a coagulation liquid, whereby a side-by-side type composite fiber is obtained.

In the present specification, the “hydrophobic modified fibroin” means a modified fibroin fiber having poor water absorbency or hygroscopicity, and the hydrophobicity may be determined, for example, by using a hydropathy index of each amino acid (hydropathy index, hereinafter also referred to as “HI”). The high hydropathy index increases the hydrophobicity of the fiber itself, and thus the shrinkage rate can be reduced even in a case where the fiber is brought into contact with an aqueous medium.

The structural protein is not particularly limited, and it may be one produced by a microorganism or the like by a genetic recombination technique, or one produced synthetically. Alternatively, the structural protein may be a purified naturally occurring structural protein.

The structural protein may be, for example, a structural protein or an artificial structural protein derived from the structural protein. The structural protein means a structural protein that forms or retains a structure and morphology in a living body. That is, the structural protein may be a naturally occurring structural protein and a modified protein in which a part of the amino acid sequence (for example, 10% or less of the amino acid sequence) is modified depending on the amino acid sequence of the naturally occurring structural protein. Examples of the structural protein include fibroin, keratin, collagen, elastin, and, resilin.

The fibroin may be, for example, one or more selected from the group consisting of a silk fibroin, a spider silk fibroin, and a hornet silk fibroin. Particularly, the structural protein may be a silk fibroin or a spider silk fibroin, or a combination thereof. In a case where the silk fibroin and the spider silk fibroin are used in combination, the proportion of the silk fibroin may be, for example, 40 parts by mass or less, 30 parts by mass or less, or 10 parts by mass or less, with respect to 100 parts by mass of the spider silk fibroin.

A silk yarn is a fiber (cocoon yarn) obtained from a cocoon made by a silkworm (Bombyx mori). In general, one thread of the cocoon yarn is composed of two threads of the silk fibroin and a glue substance (sericin) that covers the silk fibroin from the outside. The silk fibroin is composed of a number of fibrils. The silk fibroin is covered by four layers of sericin. Practically, a silk filament obtained by dissolving and removing the outer sericin by scouring is used for clothing A general silk yarn has a specific gravity of 1.33, an average fineness of 3.3 decitex, and a fiber length of about 1,300 to 1,500 m. The silk fibroin can be obtained by using a natural or indoor silkworm cocoon or a used or discarded silk fabric as the raw material.

The silk fibroin may be a sericin-removed silk fibroin, a sericin-unremoved silk fibroin, or a combination thereof. The sericin-removed silk fibroin is a purified silk fibroin obtained by removing sericin covering the silk fibroin and other fatty materials. The silk fibroin purified in this manner is preferably used in the form of a lyophilized powder. The sericin-unremoved silk fibroin is unpurified silk fibroin in which sericin and the like have not been removed.

The spider silk fibroin may contain a spider silk polypeptide selected from the group consisting of a natural spider silk structural protein and a polypeptide derived from the natural spider silk structural protein (an artificial spider silk structural protein).

Examples of the natural spider silk structural proteins include a large spinal canal bookmark silk structural protein, a weft protein, and a minor ampullate gland structural protein. Since the large spinal canal bookmark silk has a repeating region consisting of a crystalline region and a non-crystalline region (also referred to as an amorphous region), the large spinal canal bookmark silk has high stress and elasticity. The weft of the spider silk is characterized by having no crystalline region but having a repeating region consisting of the non-crystalline region. The stress of the weft is inferior to that of the large spinal canal bookmark silk, but the wet has high elasticity.

The large spinal canal bookmark silk structural protein is produced in the major ampullate gland of the spider and has a characteristic of excellent toughness. Examples of the large spinal canal bookmark silk structural protein include major ampullate spidroins MaSp1 and MaSp2 which are derived from the American golden orb-weaving spider (Nephila clavipes), and ADF3 and ADF4 which are derived from Araneus diadematus. ADF3 is one of the two major bookmark silk proteins of an orb-weaving spider. The polypeptides derived from the natural spider silk structural proteins may be polypeptides derived from these bookmark silk structural proteins. A polypeptide derived from ADF3 is relatively easy to be synthesized and has excellent properties in terms of high elongatability and toughness.

The weft structural protein is produced in the flagelliform gland of the spider. An example of the weft structural protein includes a flagellar form silk protein (flagelliform silk protein) derived from the American golden orb-weaving spider (Nephila clavipes).

The polypeptide derived from the natural spider silk structural protein may be a recombinant spider silk structural protein. Examples of the recombinant spider silk structural protein include a mutant, an analog, or a derivative of the natural spider silk structural protein. A preferred example of such a polypeptide is a recombinant spider silk structural protein (also referred to as “polypeptide derived from the large spinal canal bookmark silk structural protein”) of the large spinal canal bookmark silk protein.

An example of the structural protein derived from the large spinal canal bookmark silk and the structural protein derived from silkworm silk, which are fibroin-like structural proteins, includes a protein including a domain sequence represented by Formula 1: [(A)ₙ motif−REP1]ₘ. Here, Formula 1, A in the (A)ₙ motif represents an alanine residue, n is preferably an integer of 2 to 27 and may be an integer of 4 to 20, 8 to 20, 10 to 20, 4 to 16, 8 to 16, and 10 to 16, and the number of alanine residues may be 40% or more, 60% or more, 70% or more, 80% or more, 90% or more, and 100% (which means that the (A)ₙ motif is composed of only alanine residues), with respect to the total number of amino acid residues in the (A)ₙ motif. The REP1 represents an amino acid sequence composed of 10 to 200 amino acid residues. m represents an integer of 10 to 300. A plurality of the (A)ₙ motifs may have the same amino acid sequence or amino acid sequences different from each other. The plurality of the REP1 may have the same amino acid sequence or amino acid sequences different from each other.

The structural protein described above may be a structural protein obtained by deleting the (A)ₙ motif in Formula 1 to improve industrial productivity while maintaining strength and elongatability.

Regarding the frequency of deletion, in a case where the number of amino acid residues of the REP of two [(A)ₙ motif−REP1] units adjacent to each other is sequentially compared from the N-terminal side to the C-terminal side, then the number of amino acid residues of one REP having a small number of amino acid residues is set to 1, the maximum total value of the added numbers of amino acid residues of the described-above two [(A)ₙ motif−REP1] units adjacent to each other, in which the ratio of the number of amino acid residues of the other REP is 1.8 to 11.3, is denoted by x, and the total number of amino acid residues in the domain sequence described above is denoted by y, a structural protein, for example, having 50% or more x/y can be mentioned.

Further, the structural protein may be a structural protein having an amino acid sequence in which the content of the glycine residue is reduced, the amino acid sequence being equivalent to an amino acid sequence in which at least one or a plurality of glycine residues in the REP are substituted with other amino acid residues in the REP in Formula 1. As such a structural protein, a structural protein in which the proportion of the motif sequences in which the glycine residue is substituted with other amino acid residue is 10% or more with respect to the entire motif sequences can be mentioned.

As a specific example of the structural protein derived from the large spinal canal bookmark silk, a structural protein including the amino acid sequence set forth in SEQ ID NO: 13 and SEQ ID NO: 15 can be mentioned.

As the structural protein derived from the weft structural protein, for example, a structural protein including a domain sequence represented by Formula 2: [REP2]ₒ (Here, the REP2 in Formula 2 represents an amino acid sequence composed of Gly-Pro-Gly-Gly-X, where X represents one amino acid selected from the group composed of alanine (Ala), serine (Ser), tyrosine (Tyr), and valine (Val), and o represents an integer of 8 to 300) can be mentioned. Specifically, a structural protein including the amino acid sequence set forth in SEQ ID NO: 13 can be mentioned. The amino acid sequence represented by SEQ ID NO: 41 (PRT215) is an amino acid sequence obtained by linking an amino acid sequence (referred to as PR1 sequence), equivalent to the repeat portion and the motif, from 1,220th to 1,659th residues from the N-terminal of the partial sequence (NCBI accession No.: AAF36090, GI: 7106224) of the flagellar form silk protein of the American golden orb-weaving spider, which is obtained from the NCBI database, with a C-terminal amino acid sequence from 816th to 907th residues from the C-terminal of the partial sequence (NCBI accession No.: AAC38847, GI: 2833649) of the flagellar form silk protein of the American golden orb-weaving spider, which is obtained from the NCBI database, and further adding the amino acid sequence set forth in SEQ ID NO: 11 (a tag sequence and a hinge sequence) to the N-terminal of the linked sequence.

As a structural protein derived from collagen, for example, a structural protein including a domain sequence represented by Formula 3: [REP3]ₚ (Here, p in Formula 3 represents an integer of 5 to 300. REP3 represents an amino acid sequence composed of Gly-X-Y, where X and Y represent any amino acid residues other than Gly. A plurality of the REP3 may have the same amino acid sequence or amino acid sequences different from each other) can be mentioned. Specifically, a structural protein including the amino acid sequence set forth in SEQ ID NO: 42 can be mentioned. The amino acid sequence set forth in SEQ ID NO: 42 is obtained by adding the amino acid sequence set forth in SEQ ID NO: 11 (a tag sequence and a hinge sequence) to the N-terminal of the amino acid sequence from the 301th residue to the 540th residue, which corresponds to the repeat portion and motif of the partial sequence of human collagen type 4 (NCBI GenBank Accession No.: CAA56335.1, GI: 3702452) obtained from the NCBI database.

As the structural protein derived from resilin, for example, a structural protein including the domain sequence represented by Formula 4: [REP4]_{q} (Here, q in Formula 4 represents an integer of 4 to 300. REP4 represents an amino acid sequence composed of Ser-J-J-J-Tyr-Gly-U-Pro. J represents any amino acid residue and particularly preferably an amino acid residue selected from the group consisting of Asp, Ser, and Thr. U represents any amino acid residue and particularly preferably an amino acid residue selected from the group consisting of Pro, Ala, Thr, and Ser. A plurality of the REP4 may have the same amino acid sequence or amino acid sequences different from each other.) cab be mentioned. Specifically, a structural protein including the amino acid sequence set forth in SEQ ID NO: 43 can be mentioned. The amino acid sequence set forth in SEQ ID NO: 43 is obtained by adding the amino acid sequence set forth in SEQ ID NO: 11 (a tag sequence and a hinge sequence) to the N-terminal of the amino acid sequence from 19th residue to 321th residue of the amino acid sequence of resilin (NCBI GenBank Accession No. NP611157, Gl: 24654243), in which Thr at the 87th residue is substituted with Ser, and Asn at the 95th residue is substituted with Asp.

Examples of the structural protein derived from elastin include structural proteins having amino acid sequences such as NCBI GenBank Accession Nos., AAC98395 (human), 147076 (sheep), and NP786966 (bovine). Specifically, a structural protein including the amino acid sequence set forth in SEQ ID NO: 44 can be mentioned. The amino acid sequence set forth in SEQ ID NO: 44 is obtained by adding the amino acid sequence set forth in SEQ ID NO: 11 (a tag sequence and a hinge sequence) to the N-terminal of the amino acid sequence from 121th residue to 390th residue of the amino acid sequence of NCBI GenBank Accession No. AAC98395.

An example of the structural protein derived from keratin include a type I keratin of Capra hircus. Specifically, a structural protein including the amino acid sequence set forth in SEQ ID NO: 45 can be mentioned. The amino acid sequence set forth in SEQ ID NO: 45 (PRT798) is obtained by adding the amino acid sequence set forth in SEQ ID NO: 11 (a tag sequence and a hinge sequence) to the N-terminal of the amino acid sequence of NCBI GenBank Accession No. ACY30466.

The structural protein or a modified structural protein derived from the structural protein can be used alone or in a combination of two or more thereof. The hydrophobicity as a whole may be adjusted to the desired value by combining two or more structural proteins.

The modified fibroin may be a fibroin in which the domain sequence is different from the amino acid sequence of a naturally occurring fibroin or may be the same as the amino acid sequence of a naturally occurring fibroin. The modified fibroin used in the outermost layer may be, for example, a naturally occurring fibroin having a modified domain sequence obtained by artificially modifying a domain sequence to impart hydrophobicity.

As the specific example of the modified fibroin used in the second component, a modified fibroin having a domain sequence locally including a region having a high hydropathy index (fifth modified fibroin) or a modified fibroin having a domain sequence in which the content of the glutamine residue (sixth modified fibroin) is reduced can be mentioned. The hydropathy index of each amino acid will be described later.

The domain sequence of the fifth modified fibroin has an amino acid sequence locally including a region having a high hydropathy index, the amino acid sequence being equivalent to an amino acid sequence in which one or a plurality of amino acid residues in the REP are substituted with amino acid residues having a high hydropathy index and/or one or a plurality of amino acid residues having a high hydropathy index are inserted into the REP, as compared with a naturally occurring fibroin.

It is preferable that the region locally high in the hydropathy index is composed of two to four consecutive amino acid residues. The above-described “region having a high hydropathy index” means a region in which the sum or average of the hydropathy indices of consecutive 2 to 4 amino acid residues is higher than the sum or average of the hydropathy indices of the amino acid residues at the same position in the corresponding naturally occurring fibroin.

The “amino acid residue having a high hydropathy index” is an amino acid residue having a higher hydropathy index than the amino acid residue at the same position in the corresponding naturally occurring fibroin. It is more preferable that the above-described amino acid residues having a high hydropathy index are selected from isoleucine (I), valine (V), leucine (L), phenylalanine (F), cysteine (C), methionine (M), and alanine (A).

The fifth modified fibroin may further include the modification of the amino acid sequence equivalent to an amino acid sequence in which one or a plurality of amino acid residues are substituted, deleted, inserted and/or added, as compared with naturally occurring fibroin, in addition to the modification of the amino acid sequence in which one or a plurality of amino acid residues in the REP are substituted with amino acid residues having a high hydropathy index and/or one or a plurality of amino acid residues having a high hydropathy index are inserted into REP, as compared with naturally occurring fibroin.

The fifth modified fibroin may be obtained by, with respect to a cloned gene sequence of naturally occurring fibroin, substituting one or a plurality of hydrophilic amino acid residues in the REP (for example, amino acid residues having a negative hydropathy index) with a hydrophobic amino acid residue (for example, amino acid residues having a positive hydropathy index), and/or inserting one or a plurality of hydrophobic amino acid residues into REP. Further, for example, the modified fibroin may also be obtained by designing an amino acid sequence equivalent to an amino acid sequence in which with respect to the amino acid sequence of a naturally occurring fibroin, one or a plurality of hydrophilic amino acid residues in the REP are substituted with hydrophobic amino acid residues and/or one or a plurality of hydrophobic amino acid residues are inserted into REP, and chemically synthesizing a nucleic acid encoding the designed amino acid sequence. In any case, with respect to the amino acid sequence a naturally occurring fibroin, in addition to the modification corresponding to the substitution of one or a plurality of hydrophilic amino acid residues in the REP with hydrophobic amino acid residues and/or insertion of one or a plurality of hydrophobic amino acid residues into REP, further modification of amino acid sequence equivalent to substitution, deletion, insertion and/or addition of one or a plurality of amino acid residues may be carried out.

A fifth modified fibroin may include a domain sequence represented by Formula 1: [(A)ₙ motif−REP]ₘ and have an amino acid sequence in which p/q is 6.2% or more, in a case where in all REPs included in a sequence excluding a sequence from an (A)ₙ motif located to most C-terminal side to the C-terminal of the domain sequence from the domain sequence, the total number of amino acid residues contained in a region where an average value of hydropathy indices of four consecutive amino acid residues is 2.6 or more is denoted by p, and the total number of amino acid residues contained in the sequence excluding the sequence from the (A)ₙ motif located the most C-terminal side to the C-terminal of the domain sequence from the domain sequence is denoted by q.

Regarding the hydropathy index of amino acid residues, known indices from (Hydropathy index: Kyte J, & Doolittle R (1982)“A simple method for displaying the hydropathic character of a protein”, J. Mol. Biol., 157, pp. 105-132) may be used as a reference. Specifically, the hydropathy index (hereinafter, also referred to as “HI”) of each amino acid is as shown in Table 1 below.

TABLE 1

| Amino acid | HI |
|---|---|
| Isoleucine (Ile) | 4.5 |
| Valine (Val) | 4.2 |
| Leucine (Leu) | 3.8 |
| Phenylalanine (Phe) | 2.8 |
| Cysteine (Cys) | 2.5 |
| Methionine (Met) | 1.9 |
| Alanine (Ala) | 1.8 |
| Glycine (Gly) | −0.4 |
| Threonine (Thr) | −0.7 |
| Serine (Ser) | −0.8 |
| Tryptophan (Trp) | −0.9 |
| Tyrosine (Tyr) | −1.3 |
| Proline (Pro) | −1.6 |
| Histidine (His) | −3.2 |
| Asparagine (Asn) | −3.5 |
| Aspartic acid (Asp) | −3.5 |
| Glutamine (Gln) | −3.5 |
| Glutamic acid (Glu) | −3.5 |
| Lysine (Lys) | −3.9 |
| Arginine (Arg) | −4.5 |

The calculation method for p/q will be described in more detail. In the calculation, the sequence (hereinafter, also referred to as “sequence A”) excluding a sequence from the (A)ₙ motif located closest to the C-terminal side to the C-terminal of the domain sequence from the domain sequence represented by Formula 1: ([(A)ₙ motif−REP]ₘ−(A)ₙ motif] is used. First, in all REPs included in the sequence A, average values of hydropathy indices of the four consecutive amino acid residues are calculated. The average value of the hydropathy indices is obtained by dividing the total sum of HI of each of the amino acid residues contained in the four consecutive amino acid residues by 4 (the number of amino acid residues). The average value of the hydropathy indices is obtained for all of the four consecutive amino acid residues (each of the amino acid residues is used for calculating the average value 1 to 4 times). Next, a region where the average value of the hydropathy indices of the four consecutive amino acid residues is 2.6 or more is specified. Even in a case where a plurality of certain amino acid residues correspond to the “four consecutive amino acid residues having an average value of the hydropathy indices of 2.6 or more”, the amino acid residue is counted as one amino acid residue in the region. The total number of amino acid residues included in the region is denoted by p. The total number of amino acid residues included in the sequence A is denoted by q.

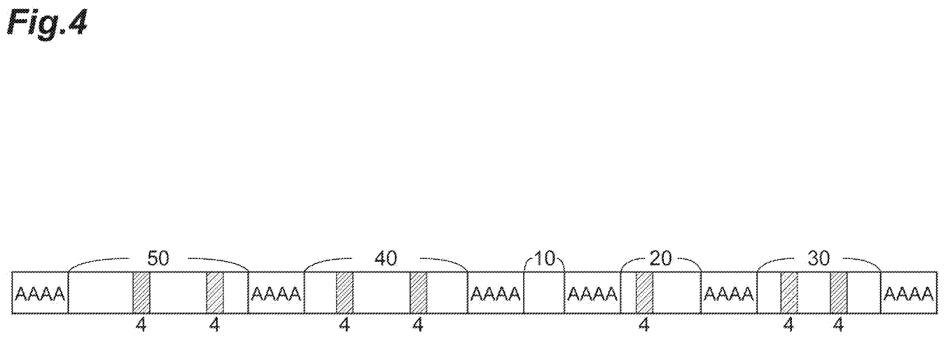

For example, in a case where the “four consecutive amino acid residues whose average value of the hydropathy indices is 2.6 or more” are extracted from 20 places (without overlap), in the region where the average value of the hydropathy indices of the four consecutive amino acid residues is 2.6 or more, the number of the four consecutive amino acid residues (without overlap) is 20, and thus p is 20×4=80. In addition, for example, in a case where two of the “four consecutive amino acid residues having an average value of the hydropathy indices of 2.6 or more” overlap by only one amino acid residue, in the region where the average value of the hydropathy indices of the four consecutive amino acid residues is 2.6 or more, the number of amino acid residues being included is 7 (p=2×4−1=7. “−1” corresponds to the subtraction of the overlapping portion). For example, in the case of the domain sequence shown in FIG. 4, since the number of the “four consecutive amino acid residues having an average value of the hydropathy indices of 2.6 or more”, which do not overlap, is 7, p is 7×4=28. Further, for example, in the case of the domain sequence illustrated in FIG. 4, q is 4+50+4+40+4+10+4+20+4+30=170 (the (A)ₙ motif present closest to the C-terminal side can not be included). Next, p/q (%) can be calculated by dividing p by q. In the case of FIG. 4, p/q (%) is 28/170=16.47%.

In the fifth modified fibroin, p/q is preferably 6.2% or more, more preferably 7% or more, still more preferably 10% or more, even still more preferably 20% or more, and still further preferably 30% or more. The upper limit of p/q is not particularly limited, but for example, it may be 45% or less.

The fifth modified fibroin may be obtained by, for example, modifying an amino acid sequence of cloned naturally occurring fibroin into an amino acid sequence locally including a region having a high hydropathy index by substituting one or a plurality of hydrophilic amino acid residues in the REP (for example, amino acid residues having a negative hydropathy index) with hydrophobic amino acid residues (for example, amino acid residues having a positive hydropathy index), and/or inserting one or a plurality of hydrophobic amino acid residues into REP, such that the p/q condition is satisfied. Alternatively, the modified fibroin may also be obtained, for example, by designing an amino acid sequence satisfying the p/q condition based on the amino acid sequence of a naturally occurring fibroin and chemically synthesizing a nucleic acid encoding the designed amino acid sequence. In any case, in addition to the modification corresponding to the substitution of one or a plurality of amino acid residues in the REP with amino acid residues having a high hydropathy index and/or insertion of one or a plurality of amino acid residues having a high hydropathy index into REP, as compared with the amino acid sequence of a naturally occurring fibroin, further modification corresponding to substitution, deletion, insertion, and/or addition of one or a plurality of amino acid residues may be carried out.

The amino acid residue having a high hydropathy index is preferably isoleucine (I), valine (V), leucine (L), phenylalanine (F), cysteine (C), methionine (M), and alanine (A), and more preferably valine (V), leucine (L), and isoleucine (I), but is not particularly limited thereto.

A more specific example of the fifth modified fibroin can include a modified fibroin including (5-i) the amino acid sequence set forth in SEQ ID NO: 19 (Met-PRT720), SEQ ID NO: 20 (Met-PRT665), or SEQ ID NO: 21 (Met-PRT666), or (5-ii) an amino acid sequence having 90% or more sequence identity with the amino acid sequence set forth in SEQ ID NO: 19, SEQ ID NO: 20, or SEQ ID NO: 21.

The modified fibroin of (5-i) will be described. The amino acid sequence set forth in SEQ ID NO: 19 is obtained by inserting an amino acid sequence consisting of three amino acid residues (VLI) at two sites for each REP with respect to the amino acid sequence set forth in SEQ ID NO: 7 (Met-PRT410), except for the end of the C-terminal side, and further substituting a part of glutamine (G) residues with serine (S) residues and deleting a part of amino acids on the C-terminal side. The amino acid sequence set forth in SEQ ID NO: 20 is obtained by inserting an amino acid sequence consisting of three amino acid residues (VLI) at one site for every other REP with respect to the amino acid sequence set forth in SEQ ID NO: 8 (Met-PRT525). The amino acid sequence set forth in SEQ ID NO: 21 is obtained by inserting an amino acid sequence consisting of three amino acid residues (VLI) at two sites for every other REP with respect to the amino acid sequence set forth in SEQ ID NO: 8.

The modified fibroin of (5-i) may consist of the amino acid sequence set forth in SEQ ID NO: 19, SEQ ID NO: 20, or SEQ ID NO: 21.

The modified fibroin of (5-ii) includes an amino acid sequence having 90% or more sequence identity with the amino acid sequence set forth in SEQ ID NO: 19, SEQ ID NO: 20, or SEQ ID NO: 21. The modified fibroin of (5-ii) is also a protein including a domain sequence represented by Formula 1: [(A)ₙ motif−REP]ₘ. The sequence identity is preferably 95% or more.

The modified fibroin of (5-ii) preferably has 90% or more sequence identity with the amino acid sequence set forth in SEQ ID NO: 19, SEQ ID NO: 20, or SEQ ID NO: 21, and preferably has an amino acid sequence in which p/q is 6.2% or more, in a case where in all REPs included in a sequence excluding a sequence from the (A)ₙ motif located closest to the C-terminal side to the C-terminal of the domain sequence from the domain sequence, the total number of amino acid residues contained in a region where an average value of hydropathy indices of the four consecutive amino acid residues is 2.6 or more is denoted by p, and the total number of amino acid residues contained in the sequence excluding a sequence from the (A)ₙ motif located closest to the C-terminal side to the C-terminal of the domain sequence from the domain sequence is denoted by q.

The fifth modified fibroin may include a tag sequence at one or both of the N-terminal and C-terminal.

A more specific example of the modified fibroin having a tag sequence can include a modified fibroin including (5-iii) the amino acid sequence set forth in SEQ ID NO: 22 (PRT720), SEQ ID NO: 23 (PRT665), or SEQ ID NO: 24 (PRT666), or (5-iv) an amino acid sequence having 90% or more sequence identity with the amino acid sequence set forth in SEQ ID NO: 22, SEQ ID NO: 23, or SEQ ID NO: 24.

The amino acid sequences set forth in SEQ ID NO: 22, SEQ ID NO: 23, and SEQ ID NO: 24 are respectively amino acid sequences obtained by adding the amino acid sequence (including a His tag sequence and a hinge sequence) set forth in SEQ ID NO: 11 to the N-terminal of the amino acid sequences set forth in SEQ ID NO: 19, SEQ ID NO: 20, and SEQ ID NO: 21.

The modified fibroin of (5-iii) may consist of the amino acid sequence set forth in SEQ ID NO: 22, SEQ ID NO: 23, or SEQ ID NO: 24.

The modified fibroin of (5-iv) includes an amino acid sequence having 90% or more sequence identity with the amino acid sequence set forth in SEQ ID NO: 22, SEQ ID NO: 23, or SEQ ID NO: 24. The modified fibroin of (5-iv) is also a protein including a domain sequence represented by Formula 1: [(A)ₙ motif−REP]ₘ. The sequence identity is preferably 95% or more.

The modified fibroin of (5-iv) preferably has 90% or more sequence identity with the amino acid sequence set forth in SEQ ID NO: 22, SEQ ID NO: 23, or SEQ ID NO: 24, and preferably has an amino acid sequence in which p/q is 6.2% or more, in a case where in all REPs included in a sequence excluding a sequence from the (A)ₙ motif located closest to the C-terminal side to the C-terminal of the domain sequence from the domain sequence, the total number of amino acid residues contained in a region where an average value of hydropathy indices of the four consecutive amino acid residues is 2.6 or more is denoted by p, and the total number of amino acid residues contained in the sequence excluding a sequence from the (A)ₙ motif located closest to the C-terminal side to the C-terminal of the domain sequence from the domain sequence is denoted by q.

The fifth modified fibroin may include a secretory signal for releasing the protein produced in the recombinant protein production system to the outside of a host. The sequence of the secretory signal can be appropriately set depending on the type of the host.

The sixth modified fibroin has an amino acid sequence with a reduced content of glutamine residue, as compared with a naturally occurring fibroin.

The sixth modified fibroin preferably includes at least one motif selected from GGX motif and GPGXX motif in the amino acid sequence of the REP.

In a case where the sixth modified fibroin includes a GPGXX motif in the REP, a GPGXX motif content rate is usually 1% or more, may be 5% or more, and is preferably 10% or more. The upper limit of the GPGXX motif content rate is not particularly limited, may be 50% or less, and may be 30% or less.

In the present specification, the “GPGXX motif content rate” is a value calculated by the following method.

In a fibroin (a modified fibroin or a naturally occurring fibroin) including a domain sequence represented by Formula 1: [(A)ₙ motif−REP]ₙ or Formula 2: [(A)ₙ motif−REP]ₘ−(A)ₙ motif, in a case where the number obtained by tripling the total number of the GPGXX motifs included in all REPs included in a sequence excluding the sequence from the (A)ₙ motif located at the most C-terminal side to the C-terminal of the domain sequence from the domain sequence (that is, equivalent to the total number of G and P in the GPGXX motifs) is denoted by s, and the total number of amino acid residues in all REPs excluding the sequence from the (A)ₙ motif located at the most the C-terminal side to the C-terminal of the domain sequence from the domain sequence and further excluding (A)ₙ motifs is denoted by t, the GPGXX motif content rate is calculated as s/t.

For the calculation of the GPGXX motif content rate, the “sequence excluding a sequence from the (A)ₙ motif located closest to the C-terminal side to the C-terminal of the domain sequence from the domain sequence” is used to exclude the effect occurring due to the fact that the “sequence from the (A)ₙ motif located closest to the C-terminal side to the C-terminal from the domain sequence” (sequence equivalent to REP) may include a sequence that is weakly correlated with the sequence characteristics of fibroin, which influences the calculation result of the GPGXX motif content rate in a case where m is small (that is, in a case where the domain sequence is short). In a case where a “GPGXX motif” is located at the C-terminal of the REP, it is treated as “GPGXX motif” even in a case where “XX” is, for example, “AA”.

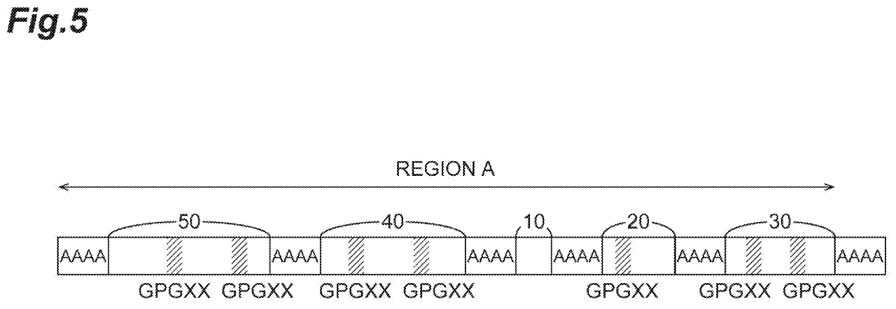

FIG. 5 is a schematic diagram showing a domain sequence of a modified fibroin. The calculation method for the GPGXX motif content rate will be specifically described with reference to FIG. 5. First, in a domain sequence (which is an [(A)ₙ motif−REP]ₘ−(A)ₙ motif] type) of a modified fibroin illustrated in FIG. 5, since all REPs are included in the “sequence excluding a sequence from the (A)ₙ motif located closest to the C-terminal side to the C-terminal of the domain sequence from the domain sequence” (in FIG. 5, shown as “region A”), the number of GPGXX motifs for calculating s is 7, and s is 7×3=21. Similarly, since all REPs are included in the “sequence excluding a sequence from the (A)ₙ motif located closest to the C-terminal side to the C-terminal of the domain sequence from the domain sequence” (in FIG. 5, shown as “region A”), t which is the total number of amino acid residues in all REPs excluding a sequence from the (A)ₙ motif located closest to the C-terminal side to the C-terminal of the domain sequence from the domain sequence and further excluding (A)ₙ motifs, is 50+40+10+20+30=150. Next, s/t (%) can be calculated by dividing s by t and is 21/150=14.0% in the case of the modified fibroin of FIG. 5.

In the sixth modified fibroin, a glutamine residue content rate is preferably 9% or less, more preferably 7% or less, still more preferably 4% or less, and particularly preferably 0%.

In the present specification, the “glutamine residue content rate” is a value calculated by the following method.

In a fibroin (a modified fibroin or a naturally occurring fibroin) including a domain sequence represented by Formula 1: [(A)ₙ motif−REP]ₘ or Formula 2: [(A)ₙ motif−REP]ₘ−(A)ₙ motif, in a case where the total number of glutamine residues included in all REPs included in a sequence (sequence equivalent to “region A” in FIG. 5) excluding the sequence from the (A)ₙ motif located at the most C-terminal side to the C-terminal of the domain sequence from the domain sequence is denoted by u, and the total number of amino acid residues in all REPs excluding the sequence from the (A)ₙ motif located at the most the C-terminal side to the C-terminal of the domain sequence from the domain sequence and further excluding (A)ₙ motifs is denoted by t, the glutamine residue content rate is calculated as u/t. For the calculation of the glutamine residue content rate, the “sequence excluding a sequence from the (A)ₙ motif located closest to the C-terminal side to the C-terminal of the domain sequence from the domain sequence” is used for the same reason described above.

The domain sequence of the sixth modified fibroin may include an amino acid sequence equivalent to an amino acid sequence in which one or a plurality of glutamine residues in the REP are deleted or substituted with other amino acid residues, as compared with a naturally occurring fibroin.

The “other amino acid residue” may be an amino acid residue other than a glutamine residue but is preferably an amino acid residue having a higher hydropathy index than that of a glutamine residue. The hydropathy indices of amino acid residues are as shown in Table 1.

As shown in Table 1, amino acid residues having a higher hydropathy index than a glutamine residue include an amino acid residue selected from isoleucine (I), valine (V), leucine (L), phenylalanine (F), cysteine (C), methionine (M), alanine (A), glycine (G), threonine (T), serine (S), tryptophan (W), tyrosine (Y), proline (P) and histidine (H). Among these, an amino acid residue selected from isoleucine (I), valine (V), leucine (L), phenylalanine (F), cysteine (C), methionine (M), and alanine (A) is more preferable, and an amino acid residue selected from isoleucine (I), valine (V), leucine (L), and phenylalanine (F) is still more preferable.

In the sixth modified fibroin, the hydrophobicity of REP is preferably −0.8 or more, more preferably −0.7 or more, still more preferably 0 or more, even still more preferably 0.3 or more, and particularly preferably 0.4 or more. The upper limit of the hydrophobicity of the REP is not particularly limited, may be 1.0 or less, and may be 0.7 or less.

In the present specification, the “hydrophobicity of REP” is a value calculated by the following method.

In a fibroin (a modified fibroin or a naturally occurring fibroin) including a domain sequence represented by Formula 1: [(A)ₙ motif−REP]ₘ or Formula 2: [(A)ₙ motif−REP]ₘ−(A)ₙ motif, in a case where the sum of the hydropathy indices of each amino acid residue included in all REPs included in a sequence (sequence equivalent to “region A” in FIG. 5) excluding the sequence from the (A)ₙ motif located at the most C-terminal side to the C-terminal of the domain sequence from the domain sequence is denoted by v, and the total number of amino acid residues in all REPs excluding the sequence from the (A)ₙ motif located at the most the C-terminal side to the C-terminal of the domain sequence from the domain sequence and further excluding (A)ₙ motifs is denoted by t, the hydrophobicity of the REP is calculated as v/t. For the calculation of the hydrophobicity of the REP, the “sequence excluding a sequence from the (A)ₙ motif located closest to the C-terminal side to the C-terminal of the domain sequence from the domain sequence” is used for the same reason described above.

The domain sequence of the sixth modified fibroin may further include an amino acid sequence equivalent to an amino acid sequence in which one or a plurality of amino acid residues are substituted, deleted, inserted and/or added, in addition to the modification of the amino acid sequence in which one or a plurality of glutamine residues in the REP are deleted and/or one or a plurality of glutamine residues in the REP are substituted with other amino acid residues, as compared with a naturally occurring fibroin.

The sixth modified fibroin can be obtained by, for example, with respect to a cloned gene sequence of a naturally occurring fibroin, deleting one or a plurality of glutamine residues in the REP and/or by substituting one or a plurality of glutamine residues in the REP with other amino acid residues. Further, for example, the modified fibroin may also be obtained by designing an amino acid sequence equivalent to an amino acid sequence in which with respect to the amino acid sequence of a naturally occurring fibroin, one or a plurality of glutamine residues in the REP are deleted and/or one or a plurality of glutamine residues in the REP are substituted with other amino acid residues, and chemically synthesizing a nucleic acid encoding the designed amino acid sequence.

A more specific example of the sixth modified fibroin can include a modified fibroin including (6-i) the amino acid sequence set forth in SEQ ID NO: 25 (Met-PRT888), SEQ ID NO: 26 (Met-PRT965), SEQ ID NO: 27 (Met-PRT889), SEQ ID NO: 28 (Met-PRT916), SEQ ID NO: 29 (Met-PRT918), SEQ ID NO: 30 (Met-PRT699), SEQ ID NO: 31 (Met-PRT698), SEQ ID NO: 32 (Met-PRT1009), or SEQ ID NO: 46 (Met-PRT966), or (6-ii) an amino acid sequence having 90% or more sequence identity with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 46.

The modified fibroin of (6-i) will be described. The amino acid sequence set forth in SEQ ID NO: 25 is obtained by substituting all QQs in the amino acid sequence set forth in SEQ ID NO: 7 (Met-PRT410) with VLs. The amino acid sequence set forth in SEQ ID NO: 26 is obtained by substituting all QQs in the amino acid sequence set forth in SEQ ID NO: 7 with TSs and substituting the remaining Qs with As. The amino acid sequence set forth in SEQ ID NO: 27 is obtained by substituting all QQs in the amino acid sequence set forth in SEQ ID NO: 7 with VLs and substituting the remaining Qs with Is. The amino acid sequence set forth in SEQ ID NO: 28 is obtained by substituting all QQs in the amino acid sequence set forth in SEQ ID NO: 7 with VIs and substituting the remaining Qs with Ls. The amino acid sequence set forth in SEQ ID NO: 29 is obtained by substituting all QQs in the amino acid sequence set forth in SEQ ID NO: 7 with VFs and substituting the remaining Qs with Is.

The amino acid sequence set forth in SEQ ID NO: 30 is obtained by substituting all QQs in the amino acid sequence set forth in SEQ ID NO: 8 (Met-PRT525) with VLs. The amino acid sequence set forth in SEQ ID NO: 31 is obtained by substituting all QQs in the amino acid sequence set forth in SEQ ID NO: 8 with VLs and substituting the remaining Qs with Is.

The amino acid sequence set forth in SEQ ID NO: 32 is an amino acid sequence obtained by repeating, four times, a region of 20 domain sequences (where several amino acid residues on the C-terminal side of the region are substituted) present in the amino acid sequence set forth in SEQ ID NO: 7 (Met-PRT410) and substituting all QQs in the repeated sequences with VFs and substituting the remaining Qs with Is.

The glutamine residue content rate of any of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32 is 9% or less (Table 2).

TABLE 2

| | Glutamine | | |
|---|---|---|---|
| | residue | GPGXX motif | Hydrophobicity |
| Modified fibroin | content rate | content rate | of REP |
| Met-PRT410 | 17.7% | 27.9% | −1.52 |
| (SEQ ID NO: 7) | | | |
| Met-PRT888 | 6.3% | 27.9% | −0.07 |
| (SEQ ID NO: 25) | | | |
| Met-PRT965 | 0.0% | 27.9% | −0.65 |
| (SEQ ID NO: 26) | | | |
| Met-PRT889 | 0.0% | 27.9% | 0.35 |
| (SEQ ID NO: 27) | | | |
| Met-PRT916 | 0.0% | 27.9% | 0.47 |
| (SEQ ID NO: 28) | | | |
| Met-PRT918 | 0.0% | 27.9% | 0.45 |
| (SEQ ID NO: 29) | | | |
| Met-PRT699 | 3.6% | 26.4% | −0.78 |
| (SEQ ID NO: 30) | | | |
| Met-PRT698 | 0.0% | 26.4% | −0.03 |
| (SEQ ID NO: 31) | | | |
| Met-PRT1009 | 0.0% | 27.9% | 0.35 |
| (SEQ ID NO: 32) | | | |

The modified fibroin of (6-i) may consist of the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 46.

The modified fibroin of (6-ii) includes an amino acid sequence having 90% or more sequence identity with the amino acid sequence set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 46. The modified fibroin of (6-ii) is also a protein including a domain sequence represented by Formula 1: [(A)ₙ motif−REP]ₘ or Formula 2: [(A)ₙ motif−REP]ₘ−(A)ₙ motif. The sequence identity is preferably 95% or more.

The modified fibroin of (6-ii) preferably has the glutamine residue content rate of 9% or less. In addition, the modified fibroin of (6-ii) preferably has the GPGXX motif content rate of 10% or more.

The sixth modified fibroin may include a tag sequence at one or both of the N-terminal and C-terminal. This makes it possible to isolate, immobilize, detect, and visualize the modified fibroin.

A more specific example of the modified fibroin having a tag sequence can include a modified fibroin including (6-iii) the amino acid sequence set forth in SEQ ID NO: 33 (PRT888), SEQ ID NO: 34 (PRT965), SEQ ID NO: 35 (PRT889), SEQ ID NO: 36 (PRT916), SEQ ID NO: 37 (PRT918), SEQ ID NO: 38 (PRT699), SEQ ID NO: 39 (PRT698), SEQ ID NO: 40 (PRT1009), or SEQ ID NO: 47 (PRT966), or (6-iv) an amino acid sequence having 90% or more sequence identity with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 47.

The amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 47 are respectively amino acid sequences obtained by adding the amino acid sequence (including a His tag sequence and a hinge sequence) set forth in SEQ ID NO: 11 to the N-terminal of the amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 46. Since only the tag sequence is added to the N-terminal, the glutamine residue content rate are not changed, and any of the amino acid sequences set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 47 has the glutamine residue content rate of 9% or less (Table 3).

TABLE 3

| | Glutamine | | |
|---|---|---|---|
| | residue | GPGXX motif | Hydrophobicity |
| Modified fibroin | content rate | content rate | of REP |
| PRT888 | 6.3% | 27.9% | −0.07 |
| (SEQ ID NO: 33) | | | |
| PRT965 | 0.0% | 27.9% | −0.65 |
| (SEQ ID NO: 34) | | | |
| PRT889 | 0.0% | 27.9% | 0.35 |
| (SEQ ID NO: 35) | | | |
| PRT916 | 0.0% | 27.9% | 0.47 |
| (SEQ ID NO: 36) | | | |
| PRT918 | 0.0% | 27.9% | 0.45 |
| (SEQ ID NO: 37) | | | |
| PRT699 | 3.6% | 26.4% | −0.78 |
| (SEQ ID NO: 38) | | | |
| PRT698 | 0.0% | 26.4% | −0.03 |
| (SEQ ID NO: 39) | | | |
| PRT1009 | 0.0% | 27.9% | 0.35 |
| (SEQ ID NO: 40) | | | |
| PRT966 | 0.0% | 28.0% | 0.35 |
| (SEQ ID NO: 47) | | | |

The modified fibroin of (6-iii) may consist of the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 47.

The modified fibroin of (6-iv) includes an amino acid sequence having 90% or more sequence identity with the amino acid sequence set forth in SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 47. The modified fibroin of (6-iv) is also a protein including a domain sequence represented by Formula 1: [(A)ₙ motif−REP]ₘ or Formula 2: [(A)ₙ motif−REP]ₘ−(A)ₙ motif. The sequence identity is preferably 95% or more.

The modified fibroin of (6-iv) preferably has the glutamine residue content rate of 9% or less. In addition, the modified fibroin of (6-iv) preferably has the GPGXX motif content rate of 10% or more.

The sixth modified fibroin may include a secretory signal for releasing the protein produced in the recombinant protein production system to the outside of a host. The sequence of the secretory signal can be appropriately set depending on the type of the host.

The limiting oxygen index (LOI) value of the modified (artificial) fibroin fiber may be 18 or more, 20 or more, 22 or more, 24 or more, 26 or more, 28 or more, 29 or more, and 30 or more. The LOT value described above is the value measured in accordance with the “testing method for powdery or low melting point synthetic resin” described in “Fire and Disaster Management Agency, Dangerous Materials Regulation Section Manager, Fire and Disaster No. 50 (on May 31, 1995)”.

The highest hygroscopic heat generation degree of the modified (artificial) fibroin fiber, which is determined according to Expression A described later, may be more than 0.025° C./g, 0.026° C./g or more, 0.027° C./g, 0.028° C./g or more, 0.029° C./g or more, 0.030° C./g or more, 0.035° C./g or more, and 0.040° C./g more. The upper limit of the highest hygroscopic heat generation degree is not particularly limited, but it is usually 0.060° C./g or less.

highest hygroscopic heat generation degree={(highest temperature of a sample when the sample has been transferred to a high humidity environment after being placed in a low humidity environment until a temperature of the sample reaches equilibrium)−(temperature of the sample when the sample is being transferred to the high humidity environment after being placed in the low humidity environment until the temperature of the sample reaches equilibrium)}(° C.)/sample weight (g) Expression A:

[In Expression A, the low humidity environment means an environment of a temperature of 20° C. and a relative humidity of 40%, and the high humidity environment means an environment of a temperature of 20° C. and a relative humidity of 90%.]

The heat retaining property index of the modified fibroin fiber may be 0.22 or more, 0.24 or more, 0.26 or more, 0.28 or more, 0.30 or more, and 0.32 or more. The upper limit of the heat retaining property index is not particularly limited, but, for example, it may be 0.60 or less or 0.40 or less.

The modified fibroin fiber preferably has excellent heat retaining property, and the heat retaining property index determined according to Formula C may be 0.20 or more.

Heat retaining property index=heat retention rate (%)/weight of sample (g/m²). Formula C:

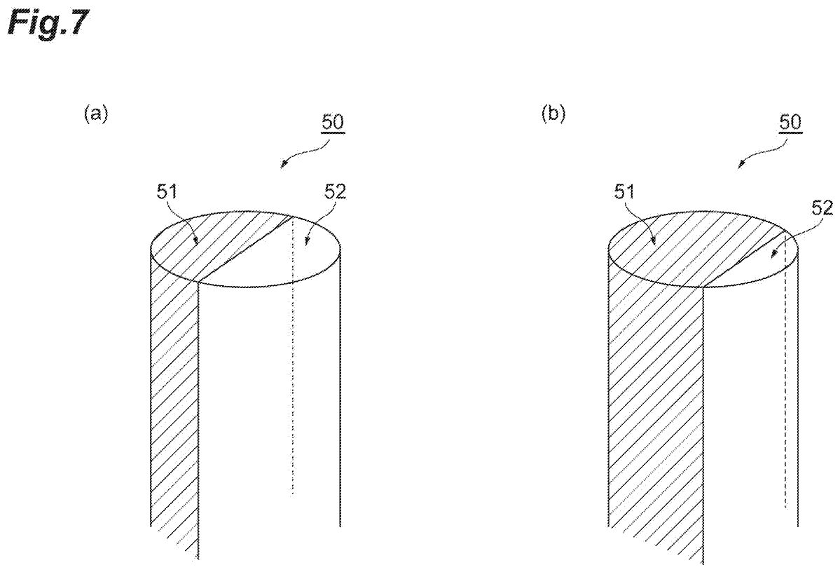

One example of a composite fiber according to one embodiment of the present invention is shown in FIG. 7. FIG. 7(a) is a schematic diagram showing a composite fiber 50 having a first component 51 and a second component 52. The first component 51 contains a modified fibroin having water shrinkability and can be shrunk by being brought into contact with an aqueous medium. On the other hand, the second component 52 has reduced water shrinkability, and the shrinkage rate due to being brought into contact with water is lower than that of the first component. FIG. 7(b) is a schematic diagram showing a composite fiber in which the configuration ratio of the first component to the second component is changed.

By appropriately changing the combination of fibers used for the first component and the second component, or changing the configuration ratio of the first component to the second component, it is possible to prepare a composite fiber having a desired crimping ability.

Due to the difference in water shrinkability between the first component and the second component, in a case where the composite fiber according to the present embodiment is brought into contact with an aqueous medium, the first component shrinks much more than the second component, and thus the crimping processing and spinning can be carried out easily. That is, the composite fiber according to the present embodiment has an excellent crimping ability and is useful as a crimped yarn or a spun yarn. The crimped yarn is excellent in spinnability, bulkiness, elasticity, flexibility, and resiliency, and can impart good feeling, soft texture, and a moisture retaining property.

The hydrophobicity of the modified fibroin contained in the first component and the hydrophobicity of the structural protein contained in the second component is different from each other. The hydrophobicity of the modified fibroin or the structural protein is a value obtained by calculating the sum of the each HI of the amino acid residues (where, the amino acid residues corresponding to the tag sequence and the hinge sequence are excluded) constituting the modified fibroin or the structural protein, and then dividing the sum of the HIs by the number of amino acid residues. The hydrophobicity of the modified fibroin contained in the first component is, for example, preferably −0.8 or less and more preferably −0.55 or less. The hydrophobicity of the structural protein contained in the second component is, for example, preferably more than −0.8 and more preferably more than −0.55.

In a case where the first component contains a plurality of modified fibroins, the hydrophobicity of each component may be obtained as an average value by calculating the hydrophobicity of each modified fibroin contained in the first component and averaging the values based on the ratio of each component. For example, a value obtained by summing the numerical values obtained by multiplying the hydrophobicity of each modified fibroin by the content rate of the modified fibroin in the first component and dividing by the number of modified fibroins may be used.

In a case where the second component contains a plurality of structural proteins, the hydrophobicity of each component may be obtained as an average value by calculating the hydrophobicity of each structural protein contained in the second component and averaging the values based on the ratio of each component. For example, a value obtained by summing the numerical values obtained by multiplying the hydrophobicity of each structural protein by the content rate of the structural protein in the second component and dividing by the number of structural proteins may be used.

Further, as described above, in a case where the first component contains a plurality of modified fibroins or in a case where the second component contains a plurality of structural proteins, the hydrophobicity of each component may be calculated without considering a modified fibroin or a structural protein contained in a low content rate (for example, a content rate of 10% or less) since the modified fibroin or the structural protein contained in a low content rate has a sufficiently small contribution to the entire hydrophobicity of the modified fibroins or the structural proteins. For example, silk yarn is composed of about 75% of silk fibroin and about 25% of sericin (UniProt database, Entry No. P07856). Silk fibroin is composed of a fibroin H chain (UniProt database, Entry No. P05790), a fibroin L chain (UniProt database, Entry No. P21828), and fibrohexamelin (UniProt database, Entry No. P04148), and the fibroin H chain quantitatively occupies most of the silk fibroin. In a case where silk fibroin of an indoor silkworm from which sericin has been removed is used as a natural structural protein, the hydrophobicity of the silk fibroin is obtained by calculating the sum of each HI of each amino acid residue of the fibroin H chain, which is the main component, and dividing the sum by the number of amino acid residues. The hydrophobicity obtained in this manner may be 0.216.

The hydrophobicity of each of the amino acid sequences set forth in SEQ ID NO: 13, SEQ ID NO: 14, SEQ ID NO: 15, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 47 is shown in Table 4. In calculating the hydrophobicity of each amino acid sequence, the calculation was performed by excluding the sequence irrelevant to the modified fibroin (that is, the sequence equivalent to the amino acid sequence represented by SEQ ID NO: 11).

TABLE 4

| Amino acid sequence | Hydrophobicity |
|---|---|
| Amino acid sequence set forth in SEQ ID NO: 13 | −0.80 |
| Amino acid sequence set forth in SEQ ID NO: 14 | −0.56 |
| Amino acid sequence set forth in SEQ ID NO: 15 | −0.80 |
| Amino acid sequence set forth in SEQ ID NO: 33 | 0.07 |
| Amino acid sequence set forth in SEQ ID NO: 34 | −0.16 |
| Amino acid sequence set forth in SEQ ID NO: 35 | 0.55 |
| Amino acid sequence set forth in SEQ ID NO: 36 | 0.54 |
| Amino acid sequence set forth in SEQ ID NO: 37 | 0.49 |
| Amino acid sequence set forth in SEQ ID NO: 38 | 0.21 |
| Amino acid sequence set forth in SEQ ID NO: 39 | 0.48 |
| Amino acid sequence set forth in SEQ ID NO: 40 | 0.49 |
| Amino acid sequence set forth in SEQ ID NO: 47 | 0.49 |

The hydrophobicity of each of the amino acid sequences represented by SEQ ID NO: 42, SEQ ID NO: 43, SEQ ID NO: 44, and SEQ ID NO: 45 is shown in Table 5. In calculating the hydrophobicity of each amino acid sequence, the calculation was performed by excluding the sequence irrelevant to the structural protein (that is, the sequence equivalent to the amino acid sequence represented by SEQ ID NO: 11).

TABLE 5

| Amino acid sequence | Hydrophobicity |
|---|---|
| Amino acid sequence set forth in SEQ ID NO: 42 | −0.74 |
| Amino acid sequence set forth in SEQ ID NO: 43 | −1.20 |
| Amino acid sequence set forth in SEQ ID NO: 44 | 0.47 |
| Amino acid sequence set forth in SEQ ID NO: 45 | −0.53 |

(Method for Producing Modified Fibroin)

A modified fibroin can be produced, for example, by expressing a nucleic acid in a host transformed with an expression vector having a nucleic acid sequence encoding the modified fibroin and one or a plurality of regulatory sequences operably linked to the nucleic acid sequence.

The method for producing a nucleic acid encoding a modified fibroin is not particularly limited. For example, the nucleic acid is produced by cloning a gene encoding the natural fibroin by amplification with polymerase chain reaction (PCR) or the like and modifying the gene by a genetic engineering method, by chemically synthesizing the nucleic acid. The method for chemically synthesizing a nucleic acid is not particularly limited, and for example, the gene can be chemically synthesized by a method in which oligonucleotides are automatically synthesized by AKTA oligopilot plus 10/100 (GE Healthcare Japan Corporation) or the like and are linked by PCR or the like, based on the amino acid sequence information of the fibroin obtained from the NCBI web database or the like. In this case, in order to facilitate purification and/or confirmation of the modified fibroin, a nucleic acid may be synthesized such that a modified fibroin having an amino acid sequence obtained by adding an amino acid sequence consisting of a start codon and a His10 tag to the N-terminal of the above amino acid sequence is encoded.

The regulatory sequence is a sequence (for example, a promoter, an enhancer, a ribosome binding sequence, or a transcription termination sequence) that controls the expression of a modified fibroin in a host, and can be appropriately selected depending on the type of the host. As a promoter, an inducible promoter that functions in a host cell and is capable of inducing the expression of a modified fibroin may be used. An inducible promoter is a promoter that can control transcription by the presence of an inducer (an expression inducer), the absence of a repressor molecule, or physical factors such as an increase or decrease in temperature, osmotic pressure, or pH value.

The type of the expression vector such as a plasmid vector, a viral vector, a cosmid vector, a fosmid vector, or an artificial chromosome vector can be appropriately selected depending on the type of the host. As the expression vector, an expression vector that can autonomously replicate in a host cell or can be incorporated into a chromosome of a host and which contains a promoter at a position capable of transcribing the nucleic acid that encodes a modified fibroin is suitably used.

Both prokaryotes and eukaryotes such as yeast, filamentous fungi, insect cells, animal cells, and plant cells can be suitably used as a host.

Preferred examples of the prokaryotic host cells include bacteria belonging to the genus Escherichia, the genus Brevibacillus, the genus Serratia, the genus Bacillus, the genus Microbacterium, the genus Brevibacterium, the genus Corynebacterium, and the genus Pseudomonas. Examples of microorganisms belonging to the genus Escherichia include Escherichia coli. Examples of the microorganisms belonging to the genus Brevibacillus include Brevibacillus agri. Examples of microorganisms belonging to the genus Serratia include Serratia liquefaciens. Examples of microorganisms belonging to the genus Bacillus include Bacillus subtilis. Examples of microorganisms belonging to the genus Microbacterium include Microbacterium ammoniaphilum. Examples of microorganisms belonging to the genus Brevibacterium include Brevibacterium divaricatum. Examples of microorganisms belonging to the genus Corynebacterium include Corynebacterium ammoniagenes. Examples of microorganisms belonging to the genus Pseudomonas include Pseudomonas putida.

In a case where a prokaryote is used as a host, examples of a vector into which a nucleic acid encoding a modified fibroin is introduced include pBTrp2 (manufactured by Boehringer Mannheim), pGEX (manufactured by Pharmacia), pUC18, pBluescriptll, pSupex, pET22b, pCold, pUB110, and pNCO2. (Japanese Unexamined Patent Publication No. 2002-238569).

Examples of eukaryotic hosts include yeast and filamentous fungi (mold and the like). Examples of yeasts include yeasts belonging to the genus Saccharomyces, the genus Pichia, and the genus Schizosaccharomyces. Examples of filamentous fungi include filamentous fungi belonging to the genus Aspergillus, the genus Penicillium, and the genus Trichoderma.

In a case where a eukaryote is used as a host, examples of the vector into which a nucleic acid encoding a modified fibroin is introduced include YEp13 (ATCC37115) and YEp24 (ATCC37051). As a method for introducing an expression vector into the above host cell, any method can be used as long as the method introduces DNA into the host cell. Examples thereof include a method using calcium ions [Proc. Natl. Acad. Sci. USA, 69, 2110 (1972)], electroporation method, spheroplast method, protoplast method, lithium acetate method, and competent method.

As for the method for expressing a nucleic acid using a host transformed with an expression vector, secretory production, fusion protein expression, or the like, in addition to the direct expression, can be carried out according to the method described in Molecular Cloning, 2nd edition.

The modified fibroin can be produced, for example, by culturing a host transformed with the expression vector in a culture medium, producing and accumulating the modified fibroin in the culture medium, and then collecting the modified fibroin from the culture medium. The method for culturing a host in a culture medium can be carried out according to a method commonly used for culturing a host.

In the case where the host is a prokaryote such as Escherichia coli or a eukaryote such as yeast, any of a natural medium and a synthetic medium may be used as a culture medium of the host as long as the medium contains a carbon source, a nitrogen source, inorganic salts and the like which can be utilized by the host and the medium can be used for efficiently culturing the host.

As the carbon source, any carbon source that can be utilized by the transformed microorganism may be used. Examples of the carbon source that can be utilized include glucose, fructose, sucrose, and molasses containing them, carbohydrates such as starch and a hydrolyzate thereof, organic acids such as acetic acid and propionic acid, and alcohols such as ethanol and propanol. Examples of the nitrogen source that can be utilized include ammonium salts of inorganic or organic acids such as ammonia, ammonium chloride, ammonium sulfate, ammonium acetate, and ammonium phosphate, other nitrogen-containing compounds, peptone, meat extract, yeast extract, corn steep liquor, casein hydrolyzate, soybean cake and soybean cake hydrolyzate, and various fermented microbial cells and digested products thereof. Examples of the inorganic salt that can be utilized include potassium dihydrogen phosphate, dipotassium phosphate, magnesium phosphate, magnesium sulfate, sodium chloride, ferrous sulfate, manganese sulfate, copper sulfate, and calcium carbonate.

Culture of a prokaryote such as Escherichia coli or a eukaryote such as yeast can be carried out under aerobic conditions such as shaking culture or deep aeration stirring culture. The culture temperature is, for example, 15° C. to 40° C. The culture time is usually 16 hours to 7 days. It is preferable to maintain the pH of the culture medium during the culture at 3.0 to 9.0. The pH of the culture medium can be adjusted using an inorganic acid, an organic acid, an alkali solution, urea, calcium carbonate, ammonia, or the like.

In addition, antibiotics such as ampicillin and tetracycline may be added to the culture medium as necessary during the culture. In a case of culturing a microorganism transformed with an expression vector using an inducible promoter as a promoter, an inducer may be added to the medium as necessary. For example, in a case of culturing a microorganism transformed with an expression vector using a lac promoter, isopropyl-β-D-thiogalactopyranoside or the like is used, and in a case of culturing a microorganism transformed with an expression vector using a trp promoter, indole acrylic acid or the like may be added to the medium.

The expressed modified fibroin can be isolated and purified by a commonly used method. For example, in a case where the modified fibroin is expressed in a dissolved state in cells, the host cells are recovered by centrifugation after the completion of the culture, suspended in an aqueous buffer solution, and then disrupted using an ultrasonicator, a French press, a Manton-Gaulin homogenizer, a Dyno-Mill, or the like to obtain a cell-free extract. From the supernatant obtained by centrifuging the cell-free extract, a purified preparation can be obtained by a method commonly used for isolation and purification of the modified fibroin, that is, a solvent extraction method, a salting-out method using ammonium sulfate or the like, a desalting method, a precipitation method using an organic solvent, an anion exchange chromatography method using a resin such as diethylaminoethyl (DEAE)-Sepharose or DIAION HPA-75 (manufactured by Mitsubishi Kasei Kogyo Kabushiki Kaisha), an cation exchange chromatography method using a resin such as S-Sepharose FF (manufacture by Pharmacia Corporation), a hydrophobic chromatography method using a resin such as butyl sepharose or phenyl sepharose, a gel filtration method using a molecular sieve, an affinity chromatography method, a chromatofocusing method, or an electrophoresis method such as isoelectric focusing or the like, using the above methods singly or in combination thereof.

In the case where the modified fibroin is expressed by the formation of an insoluble body in the cell, similarly, the host cells are recovered, disrupted and centrifuged to recover the insoluble body of the modified fibroin as a precipitated fraction. The recovered insoluble body of the modified fibroin can be solubilized with a protein denaturing agent. After this operation, a purified preparation of modified fibroin can be obtained by the same isolation and purification method as described above. In a case where the modified fibroin is secreted extracellularly, the modified fibroin can be recovered from the culture supernatant. That is, a culture supernatant is obtained by treating the culture by a technique such as centrifugation, and a purified preparation can be obtained from the culture supernatant by using the same isolation and purification method as described above.

<Doping Liquid>

A first doping liquid contains a modified fibroin and a solvent. The second doping liquid contains a structural protein and a solvent.

The concentration of the modified fibroin in the first doping liquid is not particularly limited and may be appropriately set depending on factors such as the desired crimping ability and fiber diameter of the composite fiber, and the combination with the structural protein contained in the second component. For example, the concentration of the modified fibroin is preferably 5% to 40% by mass, based on the total mass of the first doping liquid (in a case where the total mass of the first doping liquid is set to 100% by mass), more preferably 7% to 40% by mass, more preferably 10% to 40%, more preferably 7% to 35% by mass, more preferably 10% to 35% by mass, more preferably 12% to 35% by mass, more preferably 15% to 35% by mass, still more preferably 15% to 30% by mass, even still more preferably 20% to 35% by mass, and even still further preferably 20% to 30% by mass. In a case where the concentration of the modified fibroin is 5% by mass or more, the productivity of the composite fiber tends to be further improved. In a case where the concentration of the modified fibroin is 40% by mass or less, the doping liquid can be more stably discharged from the spinneret, and thus the productivity tends to be further improved.

The concentration of the structural protein in the second doping liquid is not particularly limited and may be appropriately set depending on factors such as the desired crimping ability and fiber diameter of the composite fiber, and the combination with the structural protein contained in the first component. For example, the concentration of the modified fibroin is preferably 5% to 40% by mass, based on the total mass of the second doping liquid (in a case where the total mass of the second doping liquid is set to 100% by mass), more preferably 7% to 40% by mass, more preferably 10% to 40%, more preferably 7% to 35% by mass, more preferably 10% to 35% by mass, more preferably 12% to 35% by mass, more preferably 15% to 35% by mass, still more preferably 15% to 30% by mass, even still more preferably 20% to 35% by mass, and even still further preferably 20% to 30% by mass. In a case where the concentration of the modified fibroin is 5% by mass or more, the productivity of the composite fiber tends to be further improved. In a case where the concentration of the modified fibroin is 40% by mass or less, the doping liquid can be more stably discharged from the spinneret, and thus the productivity tends to be further improved.

The solvent of the first doping liquid may be any solvent that can dissolve a modified fibroin. In addition, the solvent of the second doping liquid may be any solvent that can dissolve a structural protein. Examples of such a solvent include hexafluoroisopropanol (HFIP), hexafluoroacetone (HFA), dimethyl sulfoxide (DMSO), N,N-dimethylformamide (DMF), N,N-dimethylacetamide (DMA), 1,3-dimethyl-2-imidazolidone (DMI), N-methyl-2-pyrrolidone (NMP), acetonitrile, N-methylmorpholine-N-oxide (NMO), and formic acid. In addition, the solvent may be an aqueous solution, and specifically, an aqueous solution containing at least one selected from the group consisting of urea, guanidine, sodium dodecyl sulfate (SDS), lithium bromide, calcium chloride, and lithium thiocyanate can be mentioned. These solvents may be used alone or in a combination of two or more thereof.

The doping liquid may be prepared by a method known to those skilled in the art, and the solvent may be mixed with a modified fibroin or a structural protein in random order.

As necessary, an inorganic salt may be added to the first doping liquid and the second doping liquid. An inorganic salt may function as a dissolution accelerator of a modified fibroin. Examples of the inorganic salt include an alkali metal halide, an alkaline earth metal halide, and, an alkaline earth metal nitrate. Specific examples of the inorganic salt include lithium carbonate, lithium chloride, calcium chloride, calcium nitrate, lithium bromide, barium bromide, calcium bromide, barium chlorate, sodium perchlorate, lithium perchlorate, barium perchlorate, calcium perchlorate, and magnesium perchlorate.

The viscosity of the doping liquid can be appropriately adjusted depending on the application of the composite fiber and the spinning method. The viscosity of the doping liquid may be at 20° C., for example, 5,000 to 40,000 mPa·sec, 7,000 to 40,000 mPa·sec, 10,000 to 40,000 mPa·sec, 7,000 to 35,000 mPa·sec, 10,000 to 35,000 mPa·sec, 10,000 to 30,000 mPa·sec, or 10,000 to 25,000 mPa·sec. The viscosity of the doping liquid can be measured using, for example, an “EMS viscometer” (trade name) manufactured by Kyoto Electronics Manufacturing Co., Ltd.

The doping liquid may be stirred or shaken for some time in order to promote dissolution. At that time, heating may be performed to a temperature at which a modified fibroin or a structural protein is dissolved in the solvent. The doping liquid may be heated to, for example, 30° C. or higher, 40° C. or higher, 50° C. or higher, 60° C. or higher, 70° C. or higher, 80° C. or higher, or 90° C. or higher. The upper limit of the heating temperature is, for example, the boiling point of the solvent or lower.

<Spinning>

The composite fiber can be manufactured by a known spinning method. For example, the composite fiber can be obtained by using the first doping liquid and the second doping liquid by spinning using a known spinning method such as dry type spinning, melt spinning, wet type spinning, and dry-wet type spinning. As a preferred spinning method, wet type spinning and dry-wet type spinning can be mentioned.

In wet type spinning or dry-wet type spinning, the first doping liquid and the second doping liquid are discharged from a spinneret (nozzle) and joined to each other, and the first component (modified fibroin) and the second component (structural protein) are solidified in a coagulation liquid, whereby a composite fiber is obtained in the state of undrawn yarn.

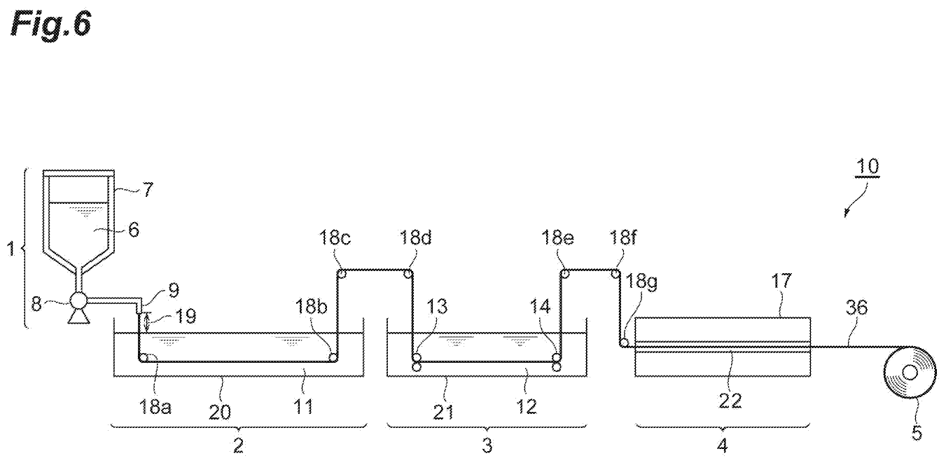

FIG. 6 is an illustrative view schematically showing an example of a spinning device for manufacturing a composite fiber. A spinning device 10 shown in FIG. 6 is an example of a spinning device for dry-wet type spinning and includes an extrusion device 1, an undrawn yarn manufacturing device 2, a wet heat drawing device 3, and a drying device 4.

A spinning method using the spinning device 10 will be described. First, a doping liquid 6 stored in a storage tank 7 is extruded out from a mouthpiece 9 by a gear pump 8. In the laboratory scale, the doping liquid may be filled in a cylinder and extruded from a nozzle using a syringe pump. Next, the extruded doping liquid 6 is supplied into a coagulation liquid 11 in a coagulation liquid bath 20 via an air gap 19, the solvent is removed, a protein is coagulated, and a fibrous coagulate is formed. Then, the fibrous coagulate is supplied into a warm water 12 in a drawing bath 21 and is drawn. A drawing rate is determined according to a speed ratio of a supply nip roller 13 to a withdrawing nip roller 14. Thereafter, the drawn fibrous coagulate is supplied to a drying device 4 and dried in a yarn path 22, and a composite fiber 36 is obtained as a wound yarn body 5. Reference signs 18a to 18g indicate yarn guides.

As the spinneret (nozzle) for manufacturing a composite fiber, for example, a nozzle for manufacturing a composite fiber having a side-by-side structure is known. In the nozzle for manufacturing a composite fiber having a side-by-side structure, a first mouthpiece for extruding the first doping liquid (corresponding to the first component of the composite fiber) is disposed in a central portion of the nozzle, and a second mouthpiece for extruding the second doping liquid (corresponding to the second component of the composite fiber) is disposed in the vicinity of the first mouthpiece. The second mouthpiece may be tilted toward the first mouthpiece such that the second doping liquid is combined with the flux of the first doping liquid extruded from the first mouthpiece. Further, it is preferable that the flux of the first mouthpiece and the flux of the second mouthpiece are parallel. The second mouthpiece may be substantially in contact with the first mouthpiece. Further, the nozzle may be one in which the first mouthpiece and the second mouthpiece are integrated and the discharge port of the first doping liquid and the discharge port of the second doping liquid are separated by a partition wall. It is preferable that each mouthpiece is designed such that the discharge is maintained at a constant amount by controlling the size and the temperature of the mouthpiece. The second doping liquid extruded from the second mouthpiece is combined with the first doping liquid extruded from the first mouthpiece to be integrated with each other, and the integrated liquids come into contact with the coagulation liquid to form an undrawn yarn having a side-by-side structure.

The position of each mouthpiece can be appropriately adjusted depending on the spinning conditions such as the type of the fiber raw material used, the viscosity of each doping liquid, the extrusion rate, the temperature, and the like.

The coagulation liquid 11 may be any liquid that can be desolvated, and examples thereof include lower alcohols having 1 to 5 carbon atoms such as methanol, ethanol, and 2-propanol, and acetone. The coagulation liquid 11 may appropriately contain water. The temperature of the coagulation liquid 11 is preferably 0° C. to 30° C. The distance that the coagulated protein passes through the coagulation liquid 11 (substantially, the distance from the yarn guide 18a to the yarn guide 18b) may be a length that allows efficient desolvation, for example, 200 to 500 mm. The retention time in the coagulation liquid 11 may be any time as long as the doping solvent is removed from the undrawn yarn. In addition, drawing (pre-drawing) may be performed in the coagulation liquid 11. The coagulation liquid bath 20 may be provided in multiple stages, and the drawing may be performed in each stage or in a specific stage as necessary. In order to suppress the evaporation of the lower alcohol, the coagulation liquid may be kept at a low temperature, and yarn may be withdrawn in an undrawn state. In addition, the undrawn yarn may be drawn in the coagulation liquid (pre-drawing).

The undrawn yarn (or pre-drawn yarn) obtained by the above method may be in the state of the drawn yarn (composite fiber) by the drawing process. As the drawing method, wet heat drawing, dry heat drawing, and the like can be mentioned.

The wet heat drawing can be performed in warm water, in a solution obtained by adding an organic solvent or the like to warm water, or in heated steam. The temperature may be, for example, may be 40° C. to 200° C., 50° C. to 180° C., 50° C. to 150° C., or 75° C. to 90° C. The drawing rate in wet heat drawing may be, for example, 1 to 30 times, 2 to 25 times, 2 to 20 times, 2 to 15 times, 2 to 10 times, 2 to 8 times, 2 to 6 times, or 2 to 4 times, with respect to the undrawn yarn (or pre-drawn yarn). However, the drawing rate is not limited as long as the desired fiber thickness and the desired mechanical properties and other characteristics can be obtained.

The dry heat drawing can be performed using a device such as a contact type hot plate and a non-contact type furnace but is not particularly limited, and any device capable of heating the fiber to the predetermined temperature and capable of drawing at the predetermined rate may be used. The temperature may be, for example, may be 100° C. to 270° C., 140° C. to 230° C., 140° C. to 200° C., 160° C. to 200° C., or 160° C. to 180° C.

The drawing rate in the dry heat drawing process may be, for example, 1 to 30 times, 2 to 30 times, 2 to 20 times, and 3 to 15 times, and is preferably 3 to 10 times, more preferably 3 to 8 times, and still more preferably 4 to 8 times, with respect to the undrawn yarn (or pre-drawn yarn). However, the drawing rate is not limited as long as the desired fiber thickness and the desired mechanical properties and other characteristics can be obtained.

In the drawing process, wet heat drawing and dry heat drawing may be carried out individually or may be carried out in multistage or in combination. That is, as the drawing process, wet heat drawing is performed as the first stage drawing and dry heat drawing is performed as the second stage drawing, or wet heat drawing is performed as the first stage drawing and wet heat drawing is performed as the second stage drawing, and then dry heat drawing may be further performed as the third stage drawing. As the drawing process, wet heat drawing and dry heat drawing may be performed appropriately combining them.

The lower limit of the final drawing rate of the composite fiber that has undergone the drawing process may be preferably 1 time, 2 times, 3 times, 4 times, 5 times, 6 times, 7 times, 8 times, and 9 times, with respect to the undrawn yarn (or pre-drawn yarn). The upper limit of the final drawing rate of the composite fiber that has undergone the drawing process may be preferably 40 times, 30 times, 20 times, 15 times, 14 times, 13 times, 12 times, 11 times, and 10 times. In addition, the final drawing rate may be, for example, 3 to 40 times, 3 to 30 times, 5 to 30 times, 5 to 20 times, 5 to 15 times, or 5 to 13 times.

In the spinning process, the mouthpiece shape, the hole shape, the number of holes, and the like of the spinneret are not particularly limited and can be appropriately selected depending on the desired fiber diameter and the number of single yarns.

Before or after the drying, an oil agent may be applied to the undrawn yarn (or pre-drawn yarn) or the drawn yarn for the purpose of imparting an antistatic property, a bundling property, lubricity and the like, as necessary. The type and amount of the oil agent to be applied are not particularly limited and can be appropriately adjusted in consideration of the application of the composite fiber, the handleability of the mating fiber, and the like.

In a case where the hole shape of the spinneret is circular, the hole diameter is, for example, 0.1 mm to 0.6 mm. In a case where the hole diameter is 0.1 mm or more, pressure loss can be reduced and equipment cost can be saved. In a case where the hole diameter is 0.6 mm or less, the drawing operation for reducing the fiber diameter can be omitted, and the possibility of rupture (drawing break) between discharging and withdrawing can be further reduced.

The temperature in a case of passing through the spinneret and the temperature of the spinneret are not particularly limited and may be appropriately adjusted depending on the concentration and viscosity of the doping liquid used, the type of solvent, and the like. The temperature of the spinneret is preferably 30° C. to 100° C. from the viewpoint of preventing the deterioration of the modified fibroin and structural proteins, and the like. In addition, the upper limit of the temperature is preferably equal to or lower than the boiling point of the solvent used, from the viewpoint of further reducing the pressure increase due to the volatilization of the solvent and the possibility of clogging inside the pipe due to the solidification of the doping liquid. This improves process stability.

The method according to the present embodiment may further include a process (filtering process) of filtering the doping liquid before discharging the doping liquid, and/or a process (defoaming process) of defoaming the doping liquid before discharging.

The crimping process is a process of crimping (hereinafter, may be referred to as “water crimping”) by bringing the composite fiber having a latent crimping ability into contact with an aqueous medium.

By bringing into contact with an aqueous medium, the composite modified fibroin fiber can be crimped without depending on the external force. The aqueous medium is a medium of a liquid or gas (steam) containing water (including steam). The aqueous medium may be water or a mixed liquid of water and a hydrophilic solvent. As the hydrophilic solvent, for example, a volatile solvent such as ethanol and methanol, or a vapor thereof can be mentioned. The aqueous medium may be a mixed liquid of water and a volatile solvent such as ethanol or methanol and is preferably water or a mixed liquid of water and ethanol. By using an aqueous medium containing a volatile solvent or a vapor thereof, it is possible to improve the drying speed after the water crimping, and furthermore impart a soft texture to a crimped staple or a crimped fiber finally obtained.

The ratio of water to the volatile solvent or the vapor thereof is not particularly limited, and, for example, water:volatile solvent may be 10:90 to 90:10 by mass ratio. The content of water is preferably 30% by mass or more and may be 40% by mass or 50% by mass or more, based on the total mass of the aqueous medium. In a case where the aqueous medium is a liquid, it is preferable to disperse an oil agent in the aqueous medium. In this case, the water crimping and the oil agent adhering can be performed at the same time. As the oil agent, any oil agent can be used as long it is a known oil agent used for general purposes including process passability and function impartability, such as an antistatic property, a friction reduction property, a flexibility imparting property, and a water repellency imparting property. The amount of the oil agent is not particularly limited and may be, for example, 1% to 10% by mass or 2% to 5% by mass with respect to the total mass of the oil agent and the aqueous medium.

The aqueous medium is preferably a liquid or gas containing water (including steam) and having a temperature of 10° C. to 230° C. The temperature of the aqueous medium may be 10° C. or higher, 25° C. or higher, 40° C. or higher, 60° C. or higher, or 100° C. or higher, and may be 230° C. or lower, 120° C. or lower, or 100° C. or lower. More specifically, in a case where the aqueous medium is a gas (steam), the temperature of the aqueous medium is preferably 100 to 230° C., more preferably 100 to 120° C. In a case where the steam of the aqueous medium is 230° C. or less, the heat denaturation of the composite fiber can be prevented. In a case where the aqueous medium is a liquid, the temperature of the aqueous medium is preferably 10° C. or higher, 25° C. or higher, or 40° C. or higher from the viewpoint of efficiently imparting crimpness, and is preferably 60° C. or lower from the viewpoint of highly maintaining fiber strength of the composite fiber.

The time of bringing into contact with the aqueous medium is not particularly limited, but may be 30 seconds or longer, 1 minute or longer, or 2 minutes or longer, and preferably 10 minutes or shorter from the viewpoint of productivity. In the case of steam, it is considered that a high shrinkage rate can be obtained in a short time in comparison with a liquid. The contact with the aqueous medium may be carried out under normal pressure or under reduced pressure (for example, vacuum).

As a method for bringing into contact with an aqueous medium, a method for immersing the composite fiber in an aqueous medium, a method for spraying the steam of an aqueous medium to the composite fiber, a method for exposing the composite fiber to an environment filled with the steam of an aqueous medium, and the like can be mentioned. In a case where the aqueous medium is steam, the composite fiber can be brought into contact with the aqueous medium by using a general steam setting device. Specific examples of the steam setting device include a device of product name: FMSA type steam setter (manufactured by Fukushin Kyougyo Co., Ltd.) and a device of a product name: EPS-400 (manufactured by Tsujii Dyeing Machine Manufacturing Co., Ltd.). As the specific example of the method for crimping the composite fiber with the steam of the aqueous medium, a method for accommodating the composite fiber in the predetermined accommodation chamber, introducing the steam of the aqueous medium into the accommodation chamber, and bring the composite fiber into contact with steam while adjusting the temperature in the accommodation chamber at the predetermined temperature (for example, 100° C. to 230° C.) described above can be mentioned.

The crimping process of the composite fiber by bringing into contact with the aqueous medium is preferably performed in a state in which no tensile force is applied to the composite fiber (no tension is applied in the fiber axis direction) or a predetermined amount of tensile force is applied (a predetermined amount of tension is applied in the fiber axis direction). At that time, it is possible to control the extent of crimping by adjusting the tensile force applied to the composite fiber. As the method for adjusting the tensile force applied to the composite fiber, for example, a method for adjusting the load applied to the fiber by hanging weights of various weights on the composite fiber, a method for identifying both ends of the fiber in the slackened state and variously changing the slackening amount, and a method for winding a fiber around a wound body such as a paper tube or bobbin and appropriately changing the winding force (tightening force on the paper tube or bobbin) at the time of winding can be mentioned.

Further, the composite fiber may be dried after being brought into contact with the aqueous medium. The drying method is not particularly limited, and the drying may be natural drying, hot storm drying, or hot roller drying. The drying temperature is not particularly limited and may be, for example, 20° C. to 150° C., preferably 40° C. to 120° C., and more preferably 60° C. to 100° C.

In the composite fiber according to the present embodiment, the first component containing a modified fibroin and the second component containing a structural protein are different in hydrophobicity with each other.

The difference in the hydrophobicity between the first component and the second component (the difference between the hydrophobicity of the modified fibroin and the hydrophobicity of the structural protein) in the composite fiber can be appropriately selected depending on the desired crimping ability, which is preferably 0.1 or more, more preferably 0.2 or more, more preferably 0.3 or more, more preferably 0.4 or more, more preferably 0.5 or more, more preferably 0.6 or more, more preferably 0.7 or more, more preferably 0.8 or more, more preferably 0.9 or more, more preferably 1.0 or more, more preferably, more preferably 1.1 or more, still more preferably 1.2, and particularly preferably 1.3 or more. The greater the hydrophobicity difference is, the more stably the latent crimping ability can be imparted.

In the composite fiber, the shrinkage rates due to water shrinkage are different between the first component and the second component. The modified fibroin used for the first component is shrunk by being brought into contact with an aqueous medium. On the other hand, the structural protein used for the second component is not shrunk even when brought into contact with an aqueous medium or has a shrinkage rate lower than that of the first component.

Table 6 shows the shrinkage rate of the modified fibroin fiber obtained by spinning the modified fibroin under the same conditions, with respect to the aqueous medium. The shrinkage rate was calculated by the following method.

<Shrinkage Rate>

A plurality of modified fibroin fibers having a length of about 30 cm are bundled to form a fiber bundle having a fineness of 150 denier. This fiber bundle, with a lead weight of 0.8 g being attached thereto, is immersed in water at 40° C. for 10 minutes to be shrunk, and the shrinkage due to the residual stress derived from the manufacturing process is removed. The fiber bundle is taken out of the water and dried at room temperature for 2 hours with 0.8 g of lead weight attached. After drying, the length of the fiber bundle is measured. Again, the fiber bundle is immersed in water at 40° C. for 10 minutes to be shrunk, and the length of the fiber bundle is measured in water. This wetting and drying are repeated at least three times, and an average length when wetted (Lwet) and an average length when dried (Ldry) are determined. The shrinkage rate is calculated according to the following expression.

Shrinkage rate (%)=(1−(Ldry/Lwet))×100 Expression:

TABLE 6

| Modified fibroin | Shrinkage rate (%) |
|---|---|
| PRT410 (SEQ ID NO: 13) | 12.0% |
| PRT888 (SEQ ID NO: 33) | 8.0% |
| PRT965 (SEQ ID NO: 34) | 8.2% |
| PRT889 (SEQ ID NO: 35) | 5.6% |
| PRT916 (SEQ ID NO: 36) | 4.2% |
| PRT918 (SEQ ID NO: 37) | 4.6% |

The composition ratio of the first component to the second component in the composite fiber is not particularly limited, and it is appropriately set depending on the combination of the first component and the second component, the desired crimping ability, the composite configuration, and the like. The composition ratio of the first component and the second component may be, for example in terms of mass, in the range of 90:10 to 10:90, in the range of 80:20 to 20:80, in the range of 75:25 to 25:75, in the range of 75:25 to 35:65, in the range of 70:30 to 30:70, in the range of 65:35 to 35:65, in the range of 65:35 to 45:55, and in the range of 60:40 to 40:60.

In a case where the composite configuration of the composite fiber is the side-by-side type, a more excellent latent crimping ability can be imparted. The transverse section of the composite fiber (the interface between the first component and the second component) may be straight or curved.

The cross-sectional shape of the composite fiber is not particularly limited, and it may be a round cross section, a triangular cross section, a multi-lobar cross section, a Daruma type cross section, a flat cross section, or any other known cross-sectional shape, but crimp developability. However, in a case where the balance of crimping exhibitability and texture is important, a cross-sectional shape such as a round cross section or a semicircular side-by-side type having a Daruma type cross section is preferred.

The fiber diameter of the composite fiber is not particularly limited, and it can be appropriately set according to the application or the like. The fiber diameter may be, for example, 10 to 125 μm, 10 to 100 μm, or 10 to 80 μm, 10 to 60 μm, 10 to 40 μm, 10 to 35 μm, or 10 to 30 μm. In a case where the fiber diameter is 125 μm or less, the desolvation rate in the spinning process is hard to be increased. In a case where the fiber diameter is 10 μm or more, the composite fiber is easy to obtained stably.

The number of crimpings of the composite fiber can be appropriately set depending on the application and the like. For example, the number of crimpings may be 5 crimpings/25 mm or more, 10 crimpings/25 mm or more, 15 crimpings/25 mm or more, 20 crimpings/25 mm or more, or 25 crimpings/25 mm or more.

Further, the composite fiber may be chemically crosslinked between polypeptide molecules in the composite fiber depending on the application and the like. Examples of functional groups that can be crosslinked include an amino group, a carboxyl group, a thiol group, and a hydroxy group. For example, an amino group of a lysine side chain contained in the polypeptide can be crosslinked through an amide bond by dehydration condensation with a carboxyl group of a glutamic acid or aspartic acid side chain. The crosslinking may be performed by performing a dehydration condensation reaction under vacuum heating, or by a dehydration condensation agent such as carbodiimides.

The crosslinking between polypeptide molecules may be performed using a crosslinking agent such as carbodiimides or glutaraldehyde, or may be performed using an enzyme such as transglutaminase Carbodiimides are compounds represented by the general formula R¹N═C═NR² (where R¹ and R² each independently represent an organic group containing an alkyl group having 1 to 6 carbon atoms or a cycloalkyl group). Specific examples of carbodiimides include 1-ethyl-3-(3-dimethylaminopropyl) carbodiimide hydrochloride (EDC), N,N′-dicyclohexylcarbodiimide (DCC), 1-cyclohexyl-3-(2-morpholinoethyl) carbodiimide, and diisopropyl carbodiimide (DIC). Among these, EDC and DIC are preferable because they have a high ability to form an amide bond between polypeptide molecules and easily perform a crosslinking reaction.

The crosslinking treatment is preferably performed by applying a crosslinking agent to the composite fiber and performing crosslinking by vacuum heating and drying. As the crosslinking agent, a pure product may be applied to the composite fiber, or a product diluted with a lower alcohol having 1 to 5 carbon atoms, a buffer solution, or the like to a concentration of 0.005 to 10 mass % may be applied to the composite fiber. The crosslinking treatment is preferably performed at a temperature of 20° C. to 45° C. for 3 to 42 hours. Higher stress (strength) can be imparted to the composite fiber by the crosslinking treatment.

(Evaluation of Crimping Ability of Composite Fiber)

The crimping ability of the composite fiber can be evaluated by, for example, checking the number of crimpings in a certain length of the composite fiber which has undergone the crimping process to exhibit a latent crimping ability.

[Product]

The composite fiber according to the present embodiment can be applied to various products. Examples of such products include fiber, yarn, fabric, knit, braid, non-woven fabric, paper, and batting. Examples of the fiber include a long fiber, a short fiber, a monofilament, and a multifilament, and examples of the yarn include a spun yarn, a twisted yarn, a pre-twisted yarn, a processed yarn, a mixed fiber yarn, and a mixed spinning yarn. Further, from these fibers and yarns, it is possible to manufacture products such as woven fabric, knit, braid, non-woven fabric, paper, batting, and the like. These products can be manufactured by a known method.

### EXAMPLES

Hereinafter, the present invention will be described more specifically based on Examples and the like. However, the present invention is not limited to the following Examples.

1. Production of Modified Fibroin and Structural Protein

(1) Preparation of Expression Vector

As the modified fibroin, modified structural proteins each having amino acid sequences set forth in SEQ ID NO: 12 (PRT380), SEQ ID NO: 13 (PRT410), SEQ ID NO: 18 (PRT399), SEQ ID NO: 15 (PRT799), SEQ ID NO: 37 (PRT918), SEQ ID NO: 47 (PRT966), and SEQ ID NO: 40 (PRT1009) (hereinafter, also referred to as respectively “PRT799”, “PRT918”, “PRT966”, and “PRT1009”) were designed, based on the base sequence and the amino acid sequence of a fibroin (GenBank accession Nos.: P46804.1, GI: 1174415) derived from Nephila clavipes. The amino acid sequence set forth in SEQ ID NO: 15 has an amino acid sequence obtained by substituting, inserting, and deleting an amino acid residue for the purpose of improving productivity with respect to the amino acid sequence of the fibroin derived from Nephila clavipes, and furthermore, adding the amino acid sequence set forth in SEQ ID NO: 11 (tag sequence and hinge sequence) to the N-terminal of the sequence. The amino acid sequence set forth in SEQ ID NO: 40 (PRT1009) is an amino acid sequence obtained by repeating, four times, a region of 20 domain sequences present in the amino acid sequence (amino acid sequence before adding the amino acid sequence set forth in SEQ ID NO: 11 to the N-terminus) set forth in SEQ ID NO: 7, substituting all QQs in the repeated sequences with VFs, and substituting the remaining Qs with Is, and furthermore, adding the amino acid sequence set forth in SEQ ID NO: 11 (tag sequence and hinge sequence) to the N-terminal of the sequence, for the purpose of improving hydrophobicity. Further, as the structural protein, a modified structural protein having the amino acid sequence set forth in SEQ ID NO: 45 (hereinafter, also referred to as “PRT798”) was designed, based on the base sequence and the amino acid sequence of a structural protein of Caora hircus (GenBank Accession No. NP001272643.1).

Next, nucleic acids encoding PRT380, PRT410, PRT399, PRT799, PRT918, PRT966, and PRT1009 (modified fibroins), and PRT798 (structural protein) were synthesized. In the nucleic acid, an NdeI site was added to the 5′ terminal and an EcoRI site was added downstream of the stop codon. The nucleic acid was cloned into a cloning vector (pUC118). Thereafter, each nucleic acid was enzymatically cleaved by treatment with NdeI and EcoRI and then recombinated into a protein expression vector pet-22b(+) to obtain an expression vector.

(2) Expression of Modified Fibroin and Structural Protein

Escherichia coli BLR(DE3) was transformed with each of the expression vectors obtained in (1) above. The above transformed Escherichia coli was cultured in 2 mL of an LB medium containing ampicillin for 15 hours. The culture solution was added to 100 mL of a seed culture medium (Table 7) containing ampicillin so that the OD₆₀₀ was 0.005. While maintaining the temperature of the culture solution at 30° C., flask culturing was carried out (for about 15 hours) until the OD₆₀₀ reached 5, thereby obtaining each seed culture solution.

TABLE 7
Seed culture medium

| Reagent | Concentration (g/L) |
|---|---|
| Glucose | 5.0 |
| KH₂PO₄ | 4.0 |
| K₂HPO₄ | 9.3 |
| Yeast Extract | 6.0 |
| Ampicillin | 0.1 |

The seed culture solution was added to a jar fermenter containing 500 mL of a production medium (Table 8) so that the OD₆₀₀ was 0.05. The culture was carried out while maintaining the temperature of culture solution at 37° C. and controlling the pH constant at 6.9. Further, the concentration of dissolved oxygen in the culture solution was maintained at 20% of the dissolved oxygen saturation concentration.

TABLE 8
Production medium

| Reagent | Concentration (g/L) |
|---|---|
| Glucose | 12.0 |
| KH₂PO₄ | 9.0 |
| MgSO₄•7H₂O | 2.4 |
| Yeast Extract | 15 |
| FeSO₄•7H₂O | 0.04 |
| MnSO₄•5H₂O | 0.04 |
| CaCl₂•2H₂O | 0.04 |
| GD-113 (anti-foaming agent) | 0.1 (mL/L) |

Immediately after glucose in the production medium was completely consumed, a feed solution (455 g/1 L of glucose and 120 g/1 L of Yeast Extract) was added at a rate of 1 mL/min. The culture was carried out while maintaining the temperature of culture solution at 37° C. and controlling the pH constant at 6.9. Further, the concentration of dissolved oxygen in the culture solution was maintained at 20% of the dissolved oxygen saturation concentration, and the culture was carried out for 20 hours. Thereafter, 1 M isopropyl-β-thiogalactopyranoside (IPTG) was added to the culture solution to a final concentration of 1 mM to induce the expression of each of the modified fibroin and the structural protein. 20 hours after the addition of IPTG, the culture solution was centrifuged to recover the bacterial cells. SDS-PAGE was carried out using bacterial cells prepared from the culture solution before the addition of IPTG and after the addition of IPTG, and the expression of each of the targeted modified fibroin and the targeted structural protein was checked by the IPTG addition-dependent appearance of a band corresponding to the size of each of the targeted modified fibroin and the targeted structural protein.

(3) Purification of Modified Fibroin and Structural Protein

The bacterial cells recovered 2 hours after the addition of IPTG were washed with a 20 mM Tris-HCl buffer solution (pH 7.4). The bacterial cells after washing were suspended in 20 mM Tris-HCl buffer solution (pH 7.4) containing about 1 mM PMSF, and the cell suspension was disrupted with a high-pressure homogenizer (manufactured by GEA Niro Soavi SpA). The disrupted cells were centrifuged to obtain a precipitate. The obtained precipitate was washed with a 20 mM Tris-HCl buffer solution (pH 7.4) until the obtained precipitate was highly pure. The precipitate after washing was suspended in 8 M guanidine buffer solution (8 M guanidine hydrochloride, 10 mM sodium dihydrogen phosphate, 20 mM NaCl, 1 mM Tris-HCl, pH 7.0) so that the concentration of the suspension was 100 mg/mL, and dissolved by stirring with a stirrer at 60° C. for 30 minutes. After dissolving, dialysis was carried out in water using a dialysis tube (cellulose tube 36/32 manufactured by Sanko Junyaku Co., Ltd.). The modified fibroins (PRT380, PRT410, PRT399, PRT799, PRT918, PRT966, and PRT1009) and the structural protein (PRT798) were obtained by recovering the white aggregated protein obtained after dialysis by centrifugation, removing the water content with a freeze dryer, and recovering the freeze-dried powder.

2. Production of Silk Fibroin (Structural Protein)

(1) Preparation of Silk Fibroin Powder

Silkworm cocoons from a natural silkworm (Bombvx mori), from which the contents therein were removed, were cut into small pieces. The pieces were boiled for about 30 minutes in boiled water containing 0.5% by mass of Marcel soap (Marcel soap finely ground with a grater was used), and then boiled for 30 minutes in boiled water. Further, this procedure was repeated two times (three times in total). Finally the pieces were boiled for 30 minutes in boiled water to completely remove sericin covering the silk fibroin, and the silk fibroin after removing sericin was dried overnight in an environment of 37° C. The dried silk was weighed, and an aqueous solution of lithium bromide (9 mol/L) was added such that the weight of the silk was 10% by mass/volume, and the silk was dissolved at 40° C. for 2 hours. This aqueous solution was put into a cellulose dialysis membrane (Seamless Cellulose Tubing, 36/32, manufactured by VISKASESELESCOAP Company), and dialyzed against distilled water for 3 to 4 days. The recovered solution after dialysis was centrifuged at 15,000 rpm and 20° C. for 1 hour to remove undissolved residues and impurities. Further, the precipitate was diluted with Milli water such that the concentration was 2% by mass or less. After the dilution, the solution was passed through a 150 μm filter manufactured by Toyo Roshi Kaisha, Ltd. to completely remove impurities. The aqueous solution of silk fibroin was frozen at −80° C. and freeze-dried overnight. By confirming that the water content was sufficiently removed, the silk fibroin powder was obtained.

3. Manufacturing and Evaluation of Composite Fiber

### Example 1

(1) Preparation of Doping Liquid

24% by mass of the modified fibroin (PRT799, hydrophobicity: −0.80) obtained in the above-described production process of the modified fibroin, as the first component, was mixed with 76% by mass of formic acid (purity 98%) as a solvent, heated with an aluminum block heater at 40° C. for 1 hour to be dissolved, filtered with a metal filter having a mesh size of 1 μm, and defoamed, whereby the first doping liquid was prepared.

The second doping liquid was prepared in the same manner as the first doping liquid, except that 24% by mass of the modified fibroin (PRT1009, hydrophobicity: 0.49) obtained in the above-described production process of the modified fibroin was used as the second component.

(2) Dry-Wet Type Spinning

Dry-wet type spinning was performed using a table-top spinning device. A reserve tank was charged with the first doping liquid and the second doping liquid, prepared as described above. While keeping the temperature at 40° C., the first doping liquid was discharged from a mono-hole nozzle having a diameter of 0.2 mm using a gear pump, then the second doping liquid was discharged from a mono-hole nozzle having the same diameter such that the second doping liquid was joined to the first doping liquid, and the joined liquids were discharged into 100% by mass of methanol in a coagulation liquid bath. After coagulation, drawing was performed in the coagulation liquid bath, further dry heat drawing was performed, followed by washing in a water washing bath to exhibit latent crimping, and a composite fiber of a side-by-side type, having a composite weight ratio of 1:1 (first component:second component), was obtained and wound.

The dry-wet type spinning conditions are as follows.

Extrusion nozzle diameter 0.2 mm

Dry heat drawing ratio: 6 times

Temperature of coagulation liquid (methanol): 5° C.

Drying temperature: 60° C.

(3) Evaluation of Crimping Ability

When the composite fiber obtained in (2) above was again subjected to a repeated process of being brought into contact with water in a water bath and then being dried, it has been confirmed that the crimping confirmed in (2) above was maintained and shown excellent bulkiness and a crimping ability.

### Example 2

(1) Preparation of Spinning Stock Solution

The first doping liquid was prepared in the same manner as in Example 1.

The second doping liquid was prepared in the same manner as in Example 1, except that 24% by mass of the structural protein (PRT798, hydrophobicity: 0.49) obtained in the above-described production process of the structural protein was used as the second component.

(2) Dry-Wet Type Spinning

A reserve tank was charged with the first doping liquid and the second doping liquid, prepared as described above. Dry-wet type spinning was performed in the same manner as in Example 1 to exhibit latent crimping, and a composite fiber of a side-by-side type, having a composite mass ratio of 1:1 (first component:second component), was obtained and wound.

(3) Evaluation of Crimping Ability

When the composite fiber obtained above was again subjected to a repeated process of being brought into contact with water in a water bath and then being dried, it has been confirmed that the crimping confirmed in (2) above was maintained and shown excellent bulkiness and a crimping ability.

### Example 3

(1) Preparation of Doping Liquid

The first doping liquid was prepared in the same manner as in Example 1.

The second doping liquid was prepared in the same manner as in Example 1, except that 24% by weight of the silk fibroin obtained in the above-described production process of the silk fibroin (structural protein) was used as the second component.

(2) Dry-Wet Type Spinning

A reserve tank was charged with the first doping liquid and the second doping liquid, prepared as described above. Dry-wet type spinning was performed in the same manner as in Example 1 to exhibit latent crimping, and a composite fiber of a side-by-side type, having a composite mass ratio of 1:1 (first component:second component), was obtained and wound.

(3) Evaluation of Crimping Ability

When the composite fiber obtained above was again subjected to a repeated process of being brought into contact with water in a water bath and then being dried, it has been confirmed that the crimping confirmed in (2) above was maintained and shown excellent bulkiness and a crimping ability.

### Reference Example 1: Combustibility Test of Modified Fibroin

A freeze-dried powder of the modified fibroin (PRT799) was added to a dimethyl sulfoxide solution of lithium chloride (concentration: 4.0% by mass) such that the concentration of the modified fibroin was 24% by mass, and mixed for 3 hours to be dissolved using a shaker. Then, insoluble bodies and bubbles were removed, whereby a modified fibroin solution (spinning stock solution) was obtained.

The obtained spinning stock solution was heated to 90° C., filtered with a metal filter having a mesh size of 5 μm, and then allowed to be left in a 30 mL stainless steel syringe to be defoamed, and then discharged from a solid nozzle having a needle diameter of 0.2 mm into 100% by mass of methanol in the coagulation bath. The discharge temperature was 90° C. After coagulation, the obtained raw yarn was wound and naturally dried to obtain a modified fibroin fiber (raw material fiber).

A knitted fabric (thickness: 180 denier, gauge number: 18) was manufactured by circular knitting using a circular knitting machine, using twisted yarn in which the raw material fibers were twisted with each other. 20 g of the obtained knitted fabric was cut out and used as a test piece.

The combustibility test was performed in accordance with the “testing method for powdery or low melting point synthetic resin” described in “Fire and Disaster No. 50 (on May 31, 1995)”. The test was performed under the conditions of a temperature of 22° C., a relative humidity of 45%, and an atmospheric pressure of 1,021 hPa. Table 9 shows the measurement results (oxygen concentration (%), combustion rate (%), and converted combustion rate (%)).

TABLE 9

| Oxygen | Combustion | Converted combustion |
|---|---|---|
| concentration (%) | rate (%) | rate (%) |
| 20.0 | 39.1 | 40.1 |
| 27.0 | 48.1 | 49.3 |
| 28.0 | 51.9 | 53.2 |
| 30.0 | 53.6 | 54.9 |
| 50.0 | 61.2 | 62.7 |
| 70.0 | 91.1 | 93.3 |
| 100.0 | 97.6 | 100.0 |

As a result of the combustibility test, the knitted fabric knitted with the modified fibroin (PRT799) fiber had a limiting oxygen index (LOI) value of 27.2. It is generally known that in a case where an LOT value is 26 or more, it is flame retardant. It can be seen that the modified fibroin has excellent flame retardancy.

### Reference Example 2: Evaluation of Hygroscopic Heat Generating Property of Modified Fibroin

A freeze-dried powder of the modified fibroin was added to a dimethyl sulfoxide solution of lithium chloride (concentration: 4.0% by mass) such that the concentration of the modified fibroin was 24% by mass, and mixed for 3 hours to be dissolved using a shaker. Then, insoluble bodies and bubbles were removed, whereby a modified fibroin solution (spinning stock solution) was obtained.

The obtained spinning stock solution was heated to 60° C., filtered with a metal filter having a mesh size of 5 μm, and then allowed to be left in a 30 mL stainless steel syringe to be defoamed, and then discharged from a solid nozzle having a needle diameter of 0.2 mm into 100% by mass of methanol in the coagulation bath. The discharge temperature was 60° C. After coagulation, the obtained raw yarn was wound and naturally dried to obtain a modified fibroin fiber (raw material fiber).

For comparison, a wool fiber, a cotton fiber, TENCEL fiber, a rayon fiber, and a polyester fiber, which are commercially available, were prepared as raw material fibers.

Each raw material fiber was used to manufacture a knitted fabric by flat knitting using a flat knitting machine. Table 10 shows the thickness and the gauge number of the knitted fabric obtained by using the PRT918 fiber or the PRT799 fiber. The thickness and gauge number of the knitted fabrics using other raw material fibers were adjusted such that the cover factor was almost the same as that of the knitted fabric of the modified fibroin fiber, which is specifically described as follows.

TABLE 10

| | | Gauge number |
|---|---|---|
| Raw material fiber | Thickness [N] | [GG] |
| PRT918 | 1/30 (wool count number | 18 |
| | single yarn) | |
| PRT799 | 1/30 (wool count number | 16 |
| | single yarn) | |
| Wool | 2/30 (two folded yarn) | 14 |
| Cotton | 2/34 (two folded yarn) | 14 |
| TENCEL | 2/30 (two folded yarn) | 15 |
| Rayon | 1/38 (single yarn) | 14 |
| Polyester | 1/60 (single yarn) | 14 |

Two pieces of knitted fabric cut into 10 cm×10 cm were overlapped together and the four sides were sewn together to obtain a test piece (sample). After leaving the test piece in a low humidity environment (temperature 20° C.±2° C., relative humidity 40%±5%) for 4 hours or more, the test piece was transferred to a high humidity environment (temperature 20° C.±2° C., relative humidity 90%±5%), and the temperature was measured at an interval of 1 minute for 30 minutes by a temperature sensor attached in the center of the inside of the test piece.

From the measurement results, the highest hygroscopic heat generation degree was determined according to Expression A.

highest hygroscopic heat generation degree={(highest temperature of a sample when the sample has been transferred to a high humidity environment after being placed in a low humidity environment until a temperature of the sample reaches equilibrium)−(temperature of the sample when the sample is being transferred to the high humidity environment after being placed in the low humidity environment until the temperature of the sample reaches equilibrium)}(° C.)/sample weight (g) Expression A:

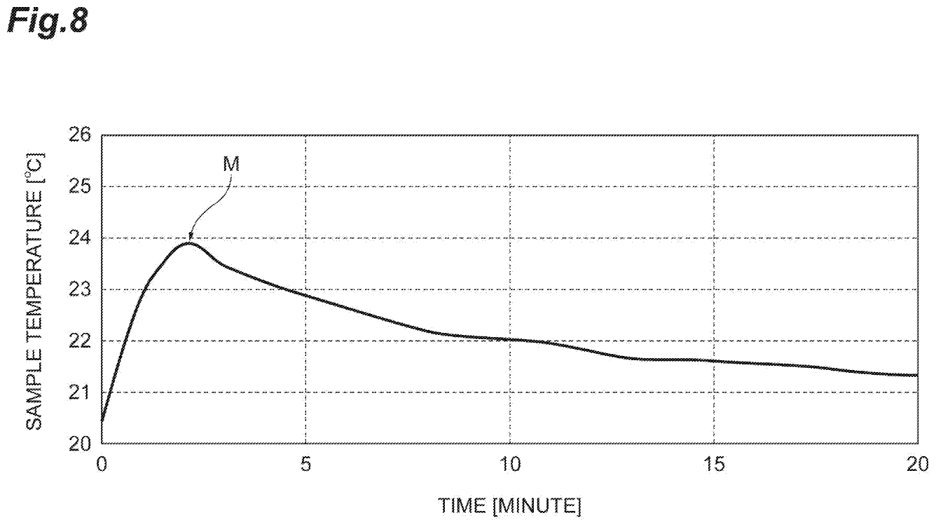

FIG. 8 is a graph showing an example of a result of a hygroscopic heat generating property test. The horizontal axis of the graph indicates the time (minutes) left in the high humidity environment, where 0 is the time when the sample was transferred from the low humidity environment to the high humidity environment. The vertical axis of the graph indicates the temperature (sample temperature) measured by the temperature sensor. In the graph shown in FIG. 8, the point indicated by M corresponds to the maximum value of the sample temperature.

Table 11 shows the calculation results of the highest hygroscopic heat generation degree of each knitted fabric.

TABLE 11

| | Highest hygroscopic heat |
|---|---|
| Raw material fiber | generation degree (° C./g) |
| PRT918 | 0.040 |
| PRT799 | 0.031 |
| Wool | 0.020 |
| Cotton | 0.021 |
| TENCEL | 0.018 |
| Rayon | 0.025 |
| Polyester | 0.010 |

As shown in Table 11, it can be seen that the modified fibroins (PRT918 and PRT799) have a higher highest hygroscopic heat generation degree and a more excellent hygroscopic heat generating property than existing materials.

### Reference Example 3: Evaluation of Heat Retaining Property of Modified Fibroin

A freeze-dried powder of the modified fibroin was added to a dimethyl sulfoxide solution of lithium chloride (concentration: 4.0% by mass) such that the concentration of the modified fibroin was 24% by mass, and mixed for 3 hours to be dissolved using a shaker. Then, insoluble bodies and bubbles were removed, whereby a modified fibroin solution (spinning stock solution) was obtained.

The obtained spinning stock solution was heated to 60° C., filtered with a metal filter having a mesh size of 5 μm, and then allowed to be left in a 30 mL stainless steel syringe to be defoamed, and then discharged from a solid nozzle having a needle diameter of 0.2 mm into 100% by mass of methanol in the coagulation bath. The discharge temperature was 60° C. After coagulation, the obtained raw yarn was wound and naturally dried to obtain a modified fibroin fiber (raw material fiber).

For comparison, a wool fiber, a silk fiber, a cotton fiber, a rayon fiber, and a polyester fiber, which are commercially available, were prepared as raw material fibers.

Each raw material fiber was used to manufacture a knitted fabric by flat knitting using a flat knitting machine. Table 12 shows the count number, number of twisted fibers, gauge number, and basis weight of the knitted fabric using the PRT966 fiber or the PRT799 fiber. The knitted fabrics using other raw material fibers were adjusted such that the cover factor was almost the same as that of the knitted fabric of the modified fibroin fiber, which is specifically described as follows.

TABLE 12

| Raw | Count | Number of | Gauge | Basis |
|---|---|---|---|---|
| material | number | twisted | number | weight |
| fiber | [Nm] | fibers | [GG] | [g/m²] |
| PRT966 | 30 | 1 | 18 | 90.1 |
| PRT799 | 30 | 1 | 16 | 111.0 |
| Wool | 30 | 2 | 14 | 242.6 |
| Silk | 60 | 2 | 14 | 225.2 |
| Cotton | 34 | 2 | 14 | 194.1 |
| Rayon | 38 | 1 | 14 | 181.8 |
| Polyester | 60 | 1 | 14 | 184.7 |

The heat retaining property was evaluated using Thermolab II tester KES-F7 manufactured by Kato Tech Co., Ltd., using a dry contact method (a method performed on assumption that skin and clothes are in direct contact with each other in a dry state). One piece of knitted fabric cut into a square of 20 cm×20 cm was used as a test piece (sample). The test piece was set on a hot plate set to a constant temperature (30° C.), and a heat quantity (a) dissipated through the test piece was obtained under the condition of the wind velocity in the wind tunnel of 30 cm/sec. A heat quantity (b) dissipated under the same condition described above was obtained without setting the test piece, and the heat retention rate (%) was calculated according to Expression B.

Heat retention rate (%)=(1−a/b)×100 Expression B:

From the measurement result, the heat retaining property index was determined according to Expression C.

Heat retaining property index=heat retention rate (%)/weight of sample (g/m²). Formula C:

Table 13 shows the calculation results of the heat retaining property index. It can be evaluated that the higher the heat retaining property index is, the more excellent the heat retaining property.

TABLE 13

| Raw material fiber | Heat retaining property index |
|---|---|
| PRT966 | 0.33 |
| PRT799 | 0.22 |
| Wool | 0.16 |
| Silk | 0.11 |
| Cotton | 0.13 |
| Rayon | 0.02 |
| Polyester | 0.18 |

As shown in Table 13, it can be seen that the modified fibroins (PRT966 and PRT799) have a higher heat retaining property index and a more excellent heat retaining property than existing materials.

### Reference Example 4: Manufacturing of Raw Material Fiber

A freeze-dried powder of the modified fibroin (PRT380, PRT410, PRT399, or PRT799) was added to a dimethyl sulfoxide solution of lithium chloride (concentration: 4.0% by mass) such that the concentration of the modified fibroin was 18% or 24% by mass (see Table 14), and mixed for 3 hours to be dissolved using a shaker. Then, insoluble bodies and bubbles were removed, whereby a modified fibroin solution (spinning stock solution) was obtained.

From the obtained spinning stock solution, raw material fibers which were spun and drawn were manufactured by a dry-wet type spinning method using a spinning device based on the spinning device 10 shown in FIG. 6. The spinning device used is a spinning device further having an additional second undrawn yarn manufacturing device (second bath) between the undrawn yarn manufacturing device 2 (first bath) and a wet heat drawing device 3 (third bath) in the spinning device 10 shown in FIG. 6. The conditions of the dry-wet type spinning method are as follows.

Extrusion nozzle diameter 0.2 mm

Liquid and temperature in the first to third baths: see Table 14

Total drawing rate: see Table 14

Drying temperature: 60° C.

TABLE 14

| | | | | | | | | | Total |
|---|---|---|---|---|---|---|---|---|---|
| | Doping liquid | | First bath | | Second bath | | Third bath | | drawing |
| | Modified | Concentration | | Temp. | | Temp. | | Temp. | rate |
| | fibroin | (% by mass) | Liquid | (° C.) | Liquid | (° C.) | Liquid | (° C.) | (times) |
| Manufacture Example 1 | PRT799 | 24 | 100% | −5 | 100% | 16 | Water | 17 | 1 |
| Manufacture Example 2 | | | Methanol | | Methanol | | | | 2 |
| Manufacture Example 3 | | | | | | | | | 3 |
| Manufacture Example 4 | | | | | | | | | 4 |
| Manufacture Example 5 | | 18 | | | | | | | 1 |
| Manufacture Example 6 | | | | | | | | | 2 |
| Manufacture Example 7 | | | | | | | | | 3 |
| Manufacture Example 8 | | | | | | | | | 4 |
| Manufacture Example 9 | PRT410 | 24 | | −11 | | 14 | | | 1 |
| Manufacture Example 10 | | | | | | | | | 2 |
| Manufacture Example 11 | | | | | | | | | 3 |
| Manufacture Example 12 | | | | | | | | | 4 |
| Manufacture Example 13 | PRT399 | | | | | | | | 1 |
| Manufacture Example 14 | | | | | | | | | 2 |
| Manufacture Example 15 | | | | | | | | | 3 |
| Manufacture Example 16 | PRT380 | | | | | 11 | | | 1 |
| Manufacture Example 17 | | | | | | | | | 2 |
| Manufacture Example 18 | | | | | | | | | 3 |
| Manufacture Example 19 | | | | | | | | | 4 |

(3) Manufacturing of Modified Fibroin Fiber and Evaluation of Shrinkage Rate a and Shrinkage Rate B

The modified fibroin fiber (artificial fibroin fiber) was manufactured by subjecting each raw material fiber obtained in Manufacture Examples 1 to 19 to the contacting step of bringing the raw material fiber into contact with water, or the drying step of drying the raw material fiber at room temperature after completion of the contacting step.

<Evaluation of Shrinkage Rate A in Contacting Step>

A plurality of raw material fiber threads having a length of 30 cm were cut from each of the wound raw material fibers obtained in Manufacture Examples 1 to 19. The plurality of raw material fiber threads were bundled to form a raw material fiber bundle having a fineness of 150 denier. With 0.8 g of lead weight being attached to each raw material fiber bundle, each raw material fiber bundle was immersed in water for 10 minutes at a temperature shown in Tables 15 to 18 (contacting step). Thereafter, the length of each raw material fiber bundle was measured in water. The length measurement of the raw material fiber bundle in water was carried out with 0.8 g lead weight being attached to the raw material fiber bundle in order to eliminate the curling of the raw material fiber bundle. Next, the shrinkage rate A (%) of each raw material fiber was calculated according to Expression (D). In Expression D, LO represents the length of the fiber before contact with water and after spinning, and here LO is 30 cm. Similarly, in Expression, Lw represents the length of the fiber irreversibly shrunk due to contact with water after spinning, and here LO is the length of each raw material fiber bundle measured in water.

Shrinkage rate A (%)={1−(Lw/LO)}×100 Expression D:

<Evaluation of Shrinkage Rate B in Drying Step>

After completion of the contacting step, the raw material fiber bundle was taken out of water. The raw material fiber bundle taken out was dried at room temperature for 2 hours with 0.8 g lead weight being attached (drying step) to obtain the modified fibroin fiber. After drying, the length of each of the modified fibroin fiber bundles was measured. Next, the shrinkage rate B (%) of each of the modified fibroin fibers was calculated according to Expression E. In Expression E, LO represents the length of the fiber before contact with water and after spinning, and here LO is 30 cm. Similarly, in Expression E, Lwd represents the length of the fiber irreversibly shrunk due to contact with water after spinning and then further shrunk by drying, and here LO is the length of each of the modified fibroin fiber bundle measured after drying.

Shrinkage rate B={1−(Lwd/LO)}×100(%) Expression E:

The results are shown in Tables 15 to 18.

TABLE 15

| | | Water | | |
|---|---|---|---|---|
| Raw material fiber/ | | temperature | Shrinkage | Shrinkage |
| artificial fibroin fiber | | (° C.) | rate A (%) | rate B (%) |
| Manufacture | 24 wt % | 20 | 0.0 | 7.8 |
| Example 1 | PRT799 | | | |
| | x1 | | | |
| Manufacture | 24 wt % | | −1.2 | 10.3 |
| Example 2 | PRT799 | | | |
| | x2 | | | |
| Manufacture | 24 wt % | | 7.2 | 21.2 |
| Example 3 | PRT799 | | | |
| | x3 | | | |
| Manufacture | 24 wt % | | 13.5 | 26.3 |
| Example 4 | PRT799 | | | |
| | x4 | | | |
| Manufacture | 18 wt % | | −2.3 | 9.5 |
| Example 6 | PRT799 | | | |
| | x2 | | | |
| Manufacture | 18 wt % | | 6.0 | 19.7 |
| Example 7 | PRT799 | | | |
| | x3 | | | |
| Manufacture | 18 wt % | | 14.3 | 27.5 |
| Example 8 | PRT799 | | | |
| | x4 | | | |
| Manufacture | 24 wt % | 40 | −5.3 | 7.2 |
| Example 2 | PRT799 | | | |
| | x2 | | | |
| Manufacture | 24 wt % | | 8.7 | 21.3 |
| Example 3 | PRT799 | | | |
| | x3 | | | |
| Manufacture | 24 wt % | | 14.5 | 26.0 |
| Example 4 | PRT799 | | | |
| | x4 | | | |
| Manufacture | 18 wt % | | −4.3 | 7.3 |
| Example 6 | PRT799 | | | |
| | x2 | | | |
| Manufacture | 18 wt % | | 6.2 | 18.3 |
| Example 7 | PRT799 | | | |
| | x3 | | | |
| Manufacture | 18 wt % | | 16.0 | 28.7 |
| Example 8 | PRT799 | | | |
| | x4 | | | |
| Manufacture | 24 wt % | 60 | 6.8 | 21.0 |
| Example 3 | PRT799 | | | |
| | x3 | | | |
| Manufacture | 24 wt % | | 15.0 | 27.5 |
| Example 4 | PRT799 | | | |
| | x4 | | | |
| Manufacture | 18 wt % | | −1.5 | 10.7 |
| Example 6 | PRT799 | | | |
| | x2 | | | |
| Manufacture | 18 wt % | | 3.3 | 18.2 |
| Example 7 | PRT799 | | | |
| | x3 | | | |
| Manufacture | 18 wt % | | 16.2 | 29.0 |
| Example 8 | PRT799 | | | |
| | x4 | | | |

TABLE 16

| | | Water | | |
|---|---|---|---|---|
| Raw material fiber/ | | temperature | Shrinkage | Shrinkage |
| artificial fibroin fiber | | (° C.) | rate A (%) | rate B (%) |
| Manufacture | 24 wt % | 20 | −2.3 | 8.7 |
| Example 10 | PRT410 | | | |
| | x2 | | | |
| Manufacture | 24 wt % | | 4.7 | 16.7 |
| Example 11 | PRT410 | | | |
| | x3 | | | |
| Manufacture | 24 wt % | | 10.3 | 22.3 |
| Example 12 | PRT410 | | | |
| | x4 | | | |
| Manufacture | 24 wt % | 40 | 4.7 | 17.5 |
| Example 11 | PRT410 | | | |
| | x3 | | | |
| Manufacture | 24 wt % | | 11.5 | 24.0 |
| Example 12 | PRT410 | | | |
| | x4 | | | |
| Manufacture | 24 wt % | 60 | 2.0 | 16.5 |
| Example 11 | PRT410 | | | |
| | x3 | | | |
| Manufacture | 24 wt % | | 10.8 | 25.0 |
| Example 12 | PRT410 | | | |
| | x4 | | | |

TABLE 17

| | | Water | | |
|---|---|---|---|---|
| Raw material fiber/ | | temperature | Shrinkage | Shrinkage |
| artificial fibroin fiber | | (° C.) | rate A (%) | rate B (%) |
| Manufacture | 24 wt % | 20 | −3.5 | 7.6 |
| Example 13 | PRT399 | | | |
| | x1 | | | |
| Manufacture | 24 wt % | | 3.7 | 12.5 |
| Example 14 | PRT399 | | | |
| | x2 | | | |
| Manufacture | 24 wt % | | 7.0 | 16.8 |
| Example 15 | PRT399 | | | |
| | x3 | | | |
| Manufacture | 24 wt % | 40 | 3.0 | 12.7 |
| Example 14 | PRT399 | | | |
| | x2 | | | |
| Manufacture | 24 wt % | | 7.3 | 16.7 |
| Example 15 | PRT399 | | | |
| | x3 | | | |
| Manufacture | 24 wt % | 60 | 3.3 | 9.3 |
| Example 14 | PRT399 | | | |
| | x2 | | | |
| Manufacture | 24 wt % | | 6.8 | 14.2 |
| Example 15 | PRT399 | | | |
| | x3 | | | |

TABLE 18

| | | Water | | |
|---|---|---|---|---|
| Raw material fiber/ | | temperature | Shrinkage | Shrinkage |
| artificial fibroin fiber | | (° C.) | rate A (%) | rate B (%) |
| Manufacture | 24 wt % | 20 | −1.1 | 9.4 |
| Example 16 | PRT380 | | | |
| | x1 | | | |
| Manufacture | 24 wt % | | 2.7 | 13.3 |
| Example 17 | PRT380 | | | |
| | x2 | | | |
| Manufacture | 24 wt % | | 7.0 | 17.7 |
| Example 18 | PRT380 | | | |
| | x3 | | | |
| Manufacture | 24 wt % | | 10.0 | 20.2 |
| Example 19 | PRT380 | | | |
| | x4 | | | |
| Manufacture | 24 wt % | 40 | 3.3 | 14.2 |
| Example 17 | PRT380 | | | |
| | x2 | | | |
| Manufacture | 24 wt % | | 7.7 | 19.0 |
| Example 18 | PRT380 | | | |
| | x3 | | | |
| Manufacture | 24 wt % | | 12.0 | 22.0 |
| Example 19 | PRT380 | | | |
| | x4 | | | |
| Manufacture | 24 wt % | 60 | 2.7 | 14.3 |
| Example 17 | PRT380 | | | |
| | x2 | | | |
| Manufacture | 24 wt % | | 8.2 | 20.3 |
| Example 18 | PRT380 | | | |
| | x3 | | | |
| Manufacture | 24 wt % | | 12.0 | 23.2 |
| Example 19 | PRT380 | | | |
| | x4 | | | |

As shown in Tables 15 to 18, it can be seen that the modified fibroin (PRT380, PRT410, PRT399, or PRT799) fiber has excellent shrinkability with respect to moisture and has an excellent latent crimping ability. In a case where a side-by-side type composite fiber is formed using the modified fibroin, a composite fiber excellent in the crimping ability, bulkiness, and elasticity can be obtained.

### REFERENCE SIGNS LIST

1 extrusion device
2 undrawn yarn manufacturing device
3 wet heat drawing device
4 drying device
6 doping liquid
9 spinneret
10 spinning device
11 coagulation liquid
20 coagulation liquid bath
21 drawing bath
36 composite fiber

1. A side-by-side type composite fiber having a latent crimping ability, comprising:
a first component containing a modified fibroin; and
a second component containing a structural protein,
wherein the first component and the second component are joined to each other.

2. The composite fiber according to claim 1, wherein a composition ratio of the first component to the second component is 9:1 to 1:9 based on a mass of the composite fiber.

3. The composite fiber according to claim 1, wherein the structural protein is at least one selected from the group consisting of a silk fibroin, a spider silk fibroin, collagen, resilin, elastin, and keratin.

4. The composite fiber according to claim 1, wherein the structural protein is at least one selected from the group consisting of a silk fibroin, a spider silk fibroin, and keratin.

5. The composite fiber according to claim 1, wherein the modified fibroin is a modified fibroin including a domain sequence represented by Formula 1: [(A)ₙ motif−REP]ₘ, and
the domain sequence has an amino acid sequence in which a content of the (A)ₙ motif is reduced, the amino acid sequence being equivalent to an amino acid sequence in which at least one or a plurality of the (A)ₙ motifs are deleted, as compared with a naturally occurring fibroin,
in Formula 1, the (A)ₙ motif represents an amino acid sequence composed of 2 to 27 amino acid residues and the number of alanine residues with respect to a total number of amino acid residues in the (A)ₙ motif is 83% or more, the REP represents an amino acid sequence composed of 10 to 200 amino acid residues, m represents an integer of 2 to 300, a plurality of the (A)ₙ motifs may have the same amino acid sequence or amino acid sequences different from each other, and a plurality of the REPs may have the same amino acid sequence or amino acid sequences different from each other.

6. The composite fiber according to claim 1, wherein the modified fibroin includes a domain sequence represented by Formula 1: [(A)ₙ motif−REP]ₘ, and
the domain sequence has an amino acid sequence in which a content of a glycine residue is reduced, the amino acid sequence being equivalent to an amino acid sequence in which at least one or a plurality of the glycine residues in the REP are substituted with other amino acid residues, as compared with a naturally occurring fibroin,
in Formula 1, the (A)ₙ motif represents an amino acid sequence composed of 2 to 27 amino acid residues and the number of alanine residues with respect to a total number of amino acid residues in the (A)ₙ motif is 83% or more, the REP represents an amino acid sequence composed of 10 to 200 amino acid residues, m represents an integer of 2 to 300, a plurality of the (A)ₙ motifs may have the same amino acid sequence or amino acid sequences different from each other, and a plurality of the REPs may have the same amino acid sequence or amino acid sequences different from each other.

7. The composite fiber according to claim 1, wherein
the modified fibroin includes a domain sequence represented by Formula 1: [(A)ₙ motif−REP]ₘ, and the domain sequence has an amino acid sequence locally including a region having a high hydropathy index, the amino acid sequence being equivalent to an amino acid sequence in which one or a plurality of amino acid residues in the REP are substituted with amino acid residues having a high hydropathy index and/or one or a plurality of amino acid residues having a high hydropathy index are inserted into the REP, as compared with a naturally occurring fibroin,
[in Formula 1, the (A)ₙ motif represents an amino acid sequence composed of 2 to 27 amino acid residues and the number of alanine residues with respect to a total number of amino acid residues in the (A)ₙ motif is 83% or more, the REP represents an amino acid sequence composed of 10 to 200 amino acid residues, m represents an integer of 2 to 300, a plurality of the (A)ₙ motifs may have the same amino acid sequence or amino acid sequences different from each other, and a plurality of the REPs may have the same amino acid sequence or amino acid sequences different from each other].

8. The composite fiber according to claim 1, wherein the modified fibroin includes a domain sequence represented by Formula 1: [(A)ₙ motif−REP]ₘ or Formula 2: [(A)ₙ motif−REP]ₘ−(A)ₙ motif, and
the domain sequence has an amino acid sequence in which a content of a glutamine residue is reduced, the amino acid sequence being equivalent to an amino acid sequence in which one or a plurality of the glutamine residues in the REP are deleted or substituted with other amino acid residues, as compared with a naturally occurring fibroin,
in Formula 1 and Formula 2, the (A)ₙ motif represents an amino acid sequence composed of 2 to 27 amino acid residues and the number of alanine residues with respect to a total number of amino acid residues in the (A)ₙ motif is 80% or more, the REP represents an amino acid sequence composed of 10 to 200 amino acid residues, m represents an integer of 2 to 300, a plurality of the (A)ₙ motifs may have the same amino acid sequence or amino acid sequences different from each other, and a plurality of the REPs may have the same amino acid sequence or amino acid sequences different from each other.

9. The composite fiber according to claim 1, wherein the modified fibroin has a limiting oxygen index (LOI) value of 26.0 or more.

10. The composite fiber according to claim 1, wherein the modified fibroin has a highest hygroscopic heat generation degree of more than 0.025° C./g, the highest hygroscopic heat generation degree being determined according to Expression A,
highest hygroscopic heat generation degree={(highest temperature of a sample when the sample has been transferred to a high humidity environment after being placed in a low humidity environment until a temperature of the sample reaches equilibrium)−(temperature of the sample when the sample is being transferred to the high humidity environment after being placed in the low humidity environment until the temperature of the sample reaches equilibrium)}(° C.)/sample weight (g), Expression A:
in Expression A, the low humidity environment means an environment of a temperature of 20° C. and a relative humidity of 40%, and the high humidity environment means an environment of a temperature of 20° C. and a relative humidity of 90%.

11-24. (canceled)

25. A side-by-side type composite fiber comprising:
a first component; and
a second component,
wherein the first component and the second component are joined to each other, and one of the first component and the second component contains a modified fibroin.

26. A side-by-side type composite fiber comprising:
a first component; and
a second component,
wherein the first component and the second component are joined to each other, and one of the first component and the second component contains a modified fibroin having a latent crimping ability.
